(19) United States
(12) Patent Application Publication
(10) Pub. No.: US 2026/0167690 A1
(43) Pub. Date: Jun. 18, 2026

(54) Unique Interferon Alpha 2B And New Method Of Making Thereof

(71) Applicant: PharmaEssentia Corporation, Taipei (TW)

(72) Inventors: Ko-Chung Lin, Lexington, MA (US); Ming-Pin Hsu, Taipei (TW); Yu-Kuei Tsai, Taipei (TW); Yi-Te Yo, Taipei (TW); Jiing-Guang Chuang, Taipei (TW); Shin-Jye Lee, Taipei (TW)

(73) Assignee: PharmaEssentia Corporation, Taipei (TW)

(21) Appl. No.: 19/361,525

(22) Filed: Oct. 17, 2025

Related U.S. Application Data
(60) Provisional application No. 63/709,816, filed on Oct. 21, 2024.
(60) Provisional application No. 63/709,821, filed on Oct. 21, 2024.
(60) Provisional application No. 63/709,823, filed on Oct. 21, 2024.
(60) Provisional application No. 63/709,850, filed on Oct. 21, 2024.
(60) Provisional application No. 63/709,859, filed on Oct. 21, 2024.

(51) Int. Cl. C07K 14/56; A61K 38/00; C07K 1/30; C07K 1/34; G01N 33/68

(52) U.S. Cl. CPC C07K 14/56; C07K 1/30; C07K 1/34; G01N 33/6866; A61K 38/00; G01N 2333/56

(57) ABSTRACT

A composition and a new method of manufacturing and producing such composition including modified interferon alpha 2 b, modified acetylated interferon alpha 2 b, and/or a mixture thereof is disclosed herein.

### CROSS-REFERENCE TO RELATED APPLICATIONS

The present application claims the benefit to U.S. Provisional Patent Application Ser. Nos. 63/709,816, 63/709,821, 63/709,823, 63/709,850, and 63/709,859 all of which filed on Oct. 21, 2024, and the content of all of which are incorporated herein by reference in their entirety.

### SEQUENCE LISTING

This application is being filed electronically via EFS-Web and includes an electronically submitted sequence listing in .xml format in ST.26 format. The .xml file contains a sequence listing entitled “038-PCT-SEQ-Listing.xml” created on Jun. 6, 2025, and having a size of 3,159 bytes and 4,096 bytes on disk. The sequence listing contained in this .xml file is part of the specification and is herein incorporated by reference in its entirety.

### TECHNICAL FIELD

The disclosure relates to the field of biomedicine, in particular to a unique human interferon alpha 2b, new method of making such an unique human interferon alpha 2b, and/or methods of detecting the use of such new method of making thereof.

### BACKGROUND

Interferon (IFN) was originally described more than 40 years ago as a substance that is produced upon stimulation of cells by viruses, and that has the ability to protect cells from infection with viruses of any kind, that is to interfere with viral infections. The antiviral activity of IFN was thus found to be non-specific, a fact that led to the idea that IFN might be used therapeutically against viral infections. The revolution that antibiotics had meant for bacterial infections, it was reasoned, might be paralleled by therapeutic use of IFN in viral infections. The human version is referred to as “hIFN”

The optimism regarding the potential of IFN as an antiviral therapeutic agent has not been fulfilled and for various reasons it is not until during the last decade that IFN has been established as a potent antiviral agent in chronic viral infections. In parallel with being the object of antiviral research, however, IFN has also been studied with regard to its anti-tumor properties, and it is today becoming a standard treatment in certain malignant diseases.

Although IFN was initially thought to be a single entity, later research has shown that there are multiple molecular species of IFN. Thus, there are three main classes of human IFN: s called alpha, beta and gamma interferons (IFN-alpha, IFN-beta and IFN-gamma) and a minor class called omega-IFN (IFN-(2). There are 13 genes, two of which are identical, for IFN-alpha, of which there are thus 12 subtypes, but only one gene, and no subtypes for each of IFN-beta and IFNg. IFN-alpha subtypes consist of 165 or 166, IFN-beta of 165 and IFN-gamma of 142 amino acid residues. IFN-alpha and IFN-beta were formerly called type I interferons and IFN-gamma type II or immune IFN.

The reason why there are so many subtypes of IFN-alpha remains enigmatic. However, the various subtypes of IFN-alpha vary markedly regarding their biological activities. Thus, for instance, the most pronounced antiviral activity on a molar basis is found in IFN-alpha-8, and IFN-alpha-1 has certain immunological activities that are absent among other subtypes. It therefore seems plausible that the different IFN-alpha subtypes are indeed separate cytokines which share some activities, notably the antiviral capacity, but otherwise have different functional profiles.

The present disclosure discloses a new method of making a modified hIFN-alpha 2b, where a proline is attached to the initial cysteine at the N-terminus, and/or how to detect whether such method of making was employed.

### SUMMARY

In an aspect, the present disclosure relates to a composition comprising Pro-hIFN alpha 2b, acetylated Pro-hIFN alpha 2b, and/or mixture thereof. In one aspect, the acetylated Pro-hIFN alpha 2b comprises one or more acetylated proline at position 1 (Pro 1) at the N-terminus, acetylated lysine at position 32 (Lys 32), acetylated lysine at position 50 (Lys50), acetylated lysine at position 71 (Lys 71), acetylated lysine at position 113 (Lys 113), acetylated serine at position 116 (Ser 116), acetylated lysine at position 132 (Lys132), acetylated lysine at position 135 (Lys 135), acetylated tyrosine at position 136 (Tyr 136), acetylated serine at position 137 (Ser 137), and/or any combinations thereof, of SEQ ID NO: 2. In another aspect, the acetylated Pro-hIFN alpha 2b comprises at least about two, three, four five, six, or more acetylated positions.

In one aspect, the acetylated Pro-hIFN alpha 2b comprises one or more acetylated proline at position 1 of the proline at the N-terminus, acetylated lysine at position 50, acetylated lysine at serine 116, acetylated serine at position 137, and/or any combinations thereof, of SEQ ID NO: 2. In another aspect, the acetylated Pro-hIFN alpha 2b comprises at least about two, three, or four acetylated positions.

In another aspect, the acetylated Pro-hIFN alpha 2b comprises one or more acetylated lysine positions at position 32, acetylated lysine at position 113, acetylated lysine at position 132, acetylated serine at position 137, and/or any combinations thereof, of SEQ ID NO: 2. In another aspect, the acetylated Pro-hIFN alpha 2b comprises at least about two, three, or four acetylated positions.

In one aspect, the acetylated Pro-hIFN alpha 2b comprises one or more acetylated lysine at position 71, acetylated tyrosine at position 136, and/or any combinations thereof, of SEQ ID NO: 2. In another aspect, the acetylated Pro-hIFN alpha 2b comprises at least about one or two acetylated positions.

In one aspect, the acetylated Pro-hIFN alpha 2b comprises one or more acetylated proline at position 1 of the proline at the N-terminus, acetylated lysine at position 32, acetylated lysine at position 50, acetylated lysine at position 71, acetylated lysine at position 113, acetylated serine at position 116, acetylated lysine at position 132, acetylated lysine at position 135, acetylated tyrosine at position 136, acetylated serine at position 137, and/or any combinations thereof, of SEQ ID NO: 2. In another aspect, the acetylated Pro-hIFN alpha 2b comprises at least about three acetylated positions, or four acetylated positions.

In an aspect, the acetylated Pro-hIFN alpha 2b comprises one or more acetylated lysine at position 32, acetylated lysine at position 71, acetylated lysine at position 113, acetylated lysine at position 132, acetylated lysine at position 135, acetylated tyrosine at position 136, and/or any combinations thereof of SEQ ID NO: 2. In another aspect, the acetylated Pro-hIFN alpha 2b comprises at least about three acetylated positions, or four acetylated positions.

In another aspect, the present disclosure relates to a pharmaceutical formulation comprising the composition comprises one or more acetylated proline at position 1 of the proline at the N-terminus, acetylated serine at position 116, acetylated serine at position 137, and/or acetylated lysine at position 50; and a pharmaceutically acceptable carrier.

In another aspect, the present disclosure relates to a pharmaceutical formulation comprising the composition comprises one or more selected from acetylated lysine at position 32, acetylated lysine at position 113, acetylated lysine at position 132, and/or acetylated lysine at position 135; and a pharmaceutically acceptable carrier.

In another aspect, the present disclosure relates to a pharmaceutical formulation comprising the composition comprises one or more selected from acetylated proline at position 1 of the proline at the N-terminus, acetylated lysine at position 32, acetylated lysine at position 50, acetylated lysine at position 113, acetylated serine at position 116, acetylated lysine at position 132, acetylated lysine at position 135, acetylated serine at position 137, and/or any combinations thereof, of SEQ ID NO: 2; and a pharmaceutically acceptable carrier.

Yet in another aspect, the present disclosure relates to a pharmaceutical formulation comprising the composition comprises one or more selected from acetylated proline at position 1 of the proline at the N-terminus, acetylated lysine at position 32, acetylated lysine at position 50, acetylated lysine at position 71, acetylated lysine at position 113, acetylated serine at position 116, acetylated lysine at position 132, acetylated lysine at position 135, acetylated tyrosine at position 136, acetylated serine at position 137, and/or any combinations thereof, of SEQ ID NO: 2; and a pharmaceutically acceptable carrier.

Yet in an aspect, the present disclosure relates to a pharmaceutical formulation comprising the composition comprises one or more selected from acetylated lysine at position 32, acetylated lysine at position 71, acetylated lysine at position 113, acetylated lysine at position 132, acetylated lysine at position 135, acetylated tyrosine at position 136, and/or any combinations thereof, of SEQ ID NO: 2; and a pharmaceutically acceptable carrier.

In an aspect, the overall quantification of the amount of acetylated Pro1 and Lys50 of Pro IFN A2B shows their overall quantitative relative ratio is about 2:1 or 1:2. In another aspect, the overall quantification analysis, the acetylated Pro1 and Lys50 of Pro IFN A2B shows that their overall quantitative relative ratio amount ratio is about 1:1

Yet in another aspect, the quantification amount of the acetylated Pro1, Lys50, and Ser116 Pro-hIFN alpha 2b's overall quantitative relative ratio is about 1:1:1. In another aspect, the acetylated amount of Pro1-acetylated Pro-hIFN alpha 2b to acetylated Lys50 Pro-hIFN alpha 2b to acetylated Ser116 Pro-hIFN alpha 2b's overall quantitative relative ratio is about 2:1:1 or 1:1:2.

In an aspect, an analysis of the acetylated Pro1, Lys50, Ser116, and Ser137 Pro-hIFN alpha 2b's shows their amount has an overall quantitative relative ratio of about 1:1:1:5. In another analysis, the acetylated amount of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b has an overall quantitative relative ratio of about 2:1:1:5.

In another aspect, the acetylated position of Pro-hIFN alpha 2b are located at lysing at position 32, lysine at position 113, lysine at position 132, and/or lysine at position 135. In an embodiment, the overall quantitative relative ratio amount of lysing at position 32, lysine at position 113, lysine at position 132, and lysine at position 135 is about 12:1:7.8:3. Alternatively, or additionally, the ratio can be about 4:0.3:2.6:1.

In an aspect, the acetylated position of Pro-hIFN alpha 2b are located at lysine at position 50 (Lys50), acetylated lysine at position 71 (Lys 71), acetylated lysine at position 113 (Lys 113), and acetylated tyrosine at position 136 (Tyr 136) is about 32:1:91:28. Alternative, or additionally, in aspect, the ratio of the acetylated position of Pro-hIFN alpha 2b are located at lysine at position 50 (Lys50), acetylated lysine at position 113 (Lys 113), and acetylated tyrosine at position 136 (Tyr 136) is about 1.14:3.25:1.

Yet in an aspect, the present disclosure relates to a Pro-hIFN alpha 2b wherein overall quantitative relative ratio amount of the Pro1-acetylated Pro-hIFN alpha 2b to acetylated Lys50 Pro-hIFN alpha 2b is about 2:1; wherein overall relative ratio amount of the Pro1-acetylated Pro-hIFN alpha 2b to acetylated Lys50 Pro-hIFN alpha 2b is about 1:1; wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 1:1:1; wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 2:1:1; wherein overall quantitative relative ratio of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b is about 1:1:1:5; or wherein overall quantitative relative ratio amount of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b is about 2:1:1:5.

In an aspect, the present disclosure relates to a method for effectively treating a subject in need who has myelofibrosis, polycythemia vera, essential thrombocythaemia, and/or chronic myeloid leukemia comprising administrating to the subject a therapeutically effective amount of the composition comprises two or more selected from acetylated proline at position 1 of the proline at the N-terminus, acetylated serine at position 116, acetylated serine at position 137, and acetylated lysine at position 50.

In an aspect, the acetylation of Pro-hIFN alpha 2b can be induced by adding acetic anhydride in the amount of between about 0.5-1.5 of Pro-hIFN alpha 2b: between about 0.05-0.15 Acetic anhydride in molar ratio. In an aspect, the molar ratio can be between about 0.9-1.1 of Pro-hIFN alpha 2b: between about 0.10-0.12 acetic anhydride in molar ratio.

In another aspect, Niacinamide (NAM) can be added during fermentation. Non-limiting example includes adding between about 6-8 g of NAM, such as about 6, 6.5, 7, 7.33, 7.5 and/or 8 g of NAM. In another aspect, the fermentation temperature can be elevated to about 38, 39, 40, 41 or 42° C.

In an aspect, the present disclosure relates to a method for effectively treating a subject in need who has myelofibrosis, polycythemia vera, essential thrombocythaemia, and/or chronic myeloid leukemia comprising administrating to the subject a therapeutically effective amount of the pharmaceutical formulation comprises one, two or more acetylated proline at position 1 of the proline at the N-terminus, acetylated serine at position 116, acetylated serine at position 137, acetylated lysine at position 50, acetylated lysine at position 32, acetylated lysine at position 71, acetylated lysine at position 113, acetylated lysine at position 132, acetylated lysine at position 135 acetylated tyrosine at position 136; and a pharmaceutically acceptable carrier. In an aspect, the myelofibrosis comprises idiopathic, primary, and/or early myelofibrosis. In an aspect, the polycythemia vera comprises high and/or low risk polycythemia vera. In an aspect, the composition or pharmaceutical formulation is administered to the subject as an injection. In another aspect, the composition is administered to the subject subcutaneously.

In an aspect, the present disclosure relates to method for producing the composition comprises two or more selected from acetylated proline at position 1 of the proline at the N-terminus, acetylated serine at position 116, acetylated serine at position 137, acetylated lysine at position 50, acetylated lysine at position 32, acetylated lysine at position 71, acetylated lysine at position 113, acetylated lysine at position 132, acetylated lysine at position 135, and/or acetylated tyrosine at position 136, where the method comprising the steps of: cloning a vector using DNA encoding the amino acid sequence of Pro-hIFN alpha 2b; transfecting said vector into a host cell; fermenting said host cell at a condition including at between about 36.9 to 37.1° C. with dissolved oxygen at about 40%, between about 40-45%, and/or above 45%; inducing Pro-hIFN alpha 2b expression with IPTG at a concentration of between about 0.50-0.9 mM; removing the initial methionine from said Pro-hIFN alpha 2b; refolding said Pro-hIFN alpha 2b; performing ultrafiltration on said Pro-hIFN protein; performing ammonium acetate precipitation on said Pro-hIFN alpha 2b; and purifying said Pro-hIFN alpha 2b. The method may further comprise the step of detecting acetylated Pro-hIFN alpha 2b. The method may further comprise the step of quantifying said acetylated Pro-hIFN alpha 2b.

In an embodiment, the Pro-hIFN alpha 2b or the acetylated from includes either denatured or folded protein.

In another aspect, additionally, or alternatively, some of the fermentation parameters to make Pro-hIFN alpha 2b can be adjusted to improve yield, as disclosed in United States Patent Application number US20250101484A1 (the contents of which are incorporated herein by reference in its entirety). For example, part of the fermentation can be performed including the steps of: carrying out seeding (time S0) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I0 (time at initiation of induction) or I1 or both; optionally, supplying a nitrogen source to any of the one or more fermenters, individually, at one or more time points selected from about S5, S6, S7, S8, S9 and S10. In an embodiment, said DO level exceeds above about 35% to 40% and said agitation rate exceeds about 300 to 700 rpm. In another aspect, said agitation rate exceeds about 500 to 600 rpm and said pH exceeds about 7.0 to 7.4. Yet in another aspect, the nitrogen source is supplied, individually, at about 10 or I1 or both, and at one or more time points selected from about S5, S6, S7, S8, S9 and S10. In an embodiment, a carbon source feeding solution is supplied at least once to at least one fermenter during the recombinant protein fermentation, wherein the carbon source feeding solution is a glucose feeding solution, fructose feeding solution, galactose feeding solution, pyruvate feeding solution, or any combination thereof. In another aspect, a cumulative total of about 800 to 1500 g of glucose is supplied to at least one fermenter, per about 5 L fermenter capacity, by the end of the recombinant protein fermentation. Yet in another aspect, the nitrogen source supplied to the one or more fermenters, individually, comprises potassium. In another aspect, the nitrogen source supplied to the one or more fermenters, individually, comprises a yeast extract, peptone, soytone, urea, tryptone, or any combinations thereof. Yet in an embodiment, about 40 to 150 mL of a yeast extract solution is supplied the one or more fermenters each time, individually; optionally about 300 to 1500 mL of a yeast extract solution is supplied to the one or more fermenters each time, individually. In another aspect, wherein the recombinant protein fermentation is performed with an inoculum ratio of about 6.0 to 8.5%. In an embodiment, said prokaryote host cell comprises an E. coli BL21, BLR-Codon Plus (DE3) cell. In another aspect, the fermentation process further comprises a step of collecting the culture of prokaryote host cell or a sample of the culture in the one or more fermenters after induction of expression of the recombinant protein, individually. In another aspect, the fermentation process further comprising processing inclusion bodies in the collected culture of prokaryote cell or sample to obtain a crude protein extract. Yet in another aspect, the culture or sample are collected after or at about 14 to 18. In an aspect, the fermentation processes the expressed recombinant protein in the crude protein extract, wherein the yield is about 5 to 10 g of the recombinant protein in a fermenter having about a 5 L capacity; optionally, wherein the yield is about 20 to 30 g of the recombinant protein in a fermenter having about a 40 L capacity.

In another aspect, the present disclosure relates to a method of detecting whether a manufacturing procedure is used to produce Pro-hIFN alpha 2b in a product, said method comprises analyzing and/or quantifying the present of acetylated Pro-hIFN alpha 2b in said product. In an aspect, the acetylated Pro-hIFN alpha 2b comprises one or more acetylated proline at position 1 of the proline at the N-terminus, acetylated serine at position 116, acetylated serine at position 137, acetylated lysine at position 50, acetylated lysine at position 32, acetylated lysine at position 71, acetylated lysine at position 113, acetylated lysine at position 132, acetylated lysine at position 135, and/or acetylated tyrosine at position 136, of said acetylated Pro-hIFN alpha. In another aspect, the acetylated Pro-hIFN alpha 2b comprises three acetylated positions, or four acetylated positions.

In another aspect, the present disclosure relates to a method of detecting whether a manufacturing procedure is used to produce Pro-hIFN alpha 2b in a product, said method comprises analyzing and/or quantifying the present of acetylated Pro-hIFN alpha 2b in said product. In an aspect, the acetylated Pro-hIFN alpha 2b comprises one or more acetylated serine at position 137, acetylated lysine at position 32, acetylated lysine at position 71, acetylated lysine at position 113, acetylated lysine at position 132, acetylated lysine at position 135, and/or acetylated tyrosine at position 136, of said acetylated Pro-hIFN alpha. In another aspect, the acetylated Pro-hIFN alpha 2b comprises three acetylated positions, or four acetylated positions.

In an aspect, the present disclosure relates to a method of detecting whether a manufacturing procedure is used to produce Pro-hIFN alpha 2b in a product, said method comprises analyzing and/or quantifying the present of acetylated Pro-hIFN alpha 2b in said product. In an aspect, the acetylated Pro-hIFN alpha 2b comprises one or more acetylated proline at acetylated serine at position 137, acetylated lysine at position 32, acetylated lysine at position 71, acetylated lysine at position 113, acetylated lysine at position 132, acetylated lysine at position 135, and/or acetylated tyrosine at position 136, of said acetylated Pro-hIFN alpha. In another aspect, the acetylated Pro-hIFN alpha 2b comprises three acetylated positions, or four acetylated positions.

Yet in another aspect, the presence of acetylated serine at position 116, acetylated serine at position 137, acetylated lysine at position 50, acetylated lysine at position 32, acetylated lysine at position 71, acetylated lysine at position 113, acetylated lysine at position 132, acetylated lysine at position 135, and/or acetylated tyrosine at position 136 of said acetylated Pro-hIFN alpha and/or mixture thereof is analyzed and/or quantified in one or more samples obtained before, during, or after one or more steps in a manufacturing procedure. In a further aspect, the presence of any of the acetylated position(s) or combinations thereof for Pro-hIFN A2b, and/or mixture thereof is analyzed and/or quantified in a products and/or said intermediates which indicates the any one of the present manufacture step(s) was utilized to produce Pro-hIFN A2b.

In another aspect, the presence of acetylated lysine at position 32, acetylated lysine at position 71, acetylated lysine at position 113, acetylated lysine at position 132, acetylated lysine at position 135, and/or acetylated tyrosine at position 136 of said acetylated Pro-hIFN alpha and/or mixture thereof is analyzed and/or quantified in one or more samples obtained before, during, or after one or more steps in a manufacturing procedure. In a further aspect, the presence of any of the acetylated position(s) or combinations thereof for Pro-hIFN A2b, and/or mixture thereof is analyzed and/or quantified in a product and/or said intermediates which indicates the any one of the present manufacture step(s) was utilized to produce Pro-hIFN A2b.

In another aspect, the presence of acetylated serine at position 116, acetylated serine at position 137, acetylated lysine at position 50, acetylated lysine at position 32, acetylated lysine at position 71, acetylated lysine at position 113, acetylated lysine at position 132, acetylated lysine at position 135, and/or acetylated tyrosine at position 136 of said acetylated Pro-hIFN alpha and/or mixture thereof is analyzed and/or quantified in one or more samples obtained before, during, or after one or more steps in a manufacturing procedure. In a further aspect, the presence of any of the acetylated position(s) or combinations thereof for Pro-hIFN A2b, and/or mixture thereof is analyzed and/or quantified from two or more individual batches at the end of a manufacturing procedure which indicates the any one of the present manufacture step(s) and/or the entire manufacturing procedure was utilized to produce Pro-hIFN A2b

In one aspect, the host cell to make the Pro-hIFN A2b in E. Coli. In another aspect, the E. Coli includes BLR-(DE 3)-RIL.

In an aspect, the present disclosure relates to the use of the composition comprises two or more selected from acetylated proline at position 1 of the proline at the N-terminus, acetylated serine at position 116, acetylated serine at position 137, acetylated lysine at position 50, acetylated lysine at position 32, acetylated lysine at position 71, acetylated lysine at position 113, acetylated lysine at position 132, acetylated lysine at position 135, acetylated tyrosine at position 136, for the preparation of a medicament for the treatment of one or more conditions including a subject having a disorder including myelofibrosis, polycythemia vera, essential thrombocythaemia, and/or chronic myeloid leukemia. The myelofibrosis may be idiopathic, primary, and/or early myelofibrosis. The polycythemia vera may be high and/or low risk polycythemia vera.

In an aspect, the present disclosure relates to a composition comprises two or more selected from acetylated proline at position 1 of the proline at the N-terminus, acetylated serine at position 116, acetylated serine at position 137, acetylated lysine at position 50, acetylated lysine at position 32, acetylated lysine at position 71, acetylated lysine at position 113, acetylated lysine at position 132, acetylated lysine at position 135 and/or acetylated tyrosine at position 136, characterized in that it is used to treating one or more of condition including a subject having a disorder including myelofibrosis, polycythemia vera, essential thrombocythaemia, and/or chronic myeloid leukemia. The composition may be characterized in that said myelofibrosis is idiopathic, primary, and/or early myelofibrosis. The composition may be characterized in that said polycythemia vera is high and/or low risk polycythemia vera.

Further in an aspect, the disclosure further elaborated that disclosed method above wherein the process of obtaining the composition further includes one or more filtration steps, ammonium sulfate and acetate precipitation, and/or anion exchange chromatography, and the pH of the composition or method of obtaining the composition is to be maintained at about 7.0 or below about 7.0.

In an aspect, not all of the acetylation species are present in a single run. In another aspect, no acetylation species are produced.

### BRIEF DESCRIPTION OF FIGURES

For a more complete understanding of the features and advantages of the present disclosure, reference is now made to the detailed description of the disclosure along with the accompanying figures and in which:

FIG. 1 denotes the amino acid sequence of the hIFN alpha 2b molecule, having 165 amino acids designated as SEQ ID NO:1.

FIG. 2 denotes the amino acid sequence of the modified hIFN alpha 2b molecule, having 166 amino acids designated as SEQ ID NO:2.

FIG. 3 denotes SDS gel electrophoresis of hIFN alpha 2b.

FIG. 4 denotes elution profile of AIX purification performed.

FIG. 5 denotes Surface Plasmon Resonance Assay for Binding Interaction between Pro-IFN alfa-2b, acetylated Pro-IFN alfa-2b, and/or mixture thereof and IFNAR1-Fc/IFNAR2-Fc. The analysis includes three steps as shown in the figure. The rate constant of association (kon) and the rate constant of dissociation (koff) are obtained from curve fitting after binding experiments in step 3.

### DETAIL DESCRIPTIONS

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity but include the general class of which a specific example may be used for illustration. The terminology herein is used to describe specific embodiments of the disclosure, but their usage does not delimit the disclosure, except as outlined in the claims.

The following terms, unless otherwise indicated, shall be understood to have the following meanings.

As used herein, “administering,” means oral administration, administration as a suppository, topical contact, intravenous, intraperitoneal, intramuscular, intralesional, intranasal or subcutaneous administration, or the implantation of a slow-release device e.g., a mini-osmotic pump, to the subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

As used herein, the term “isolated molecule” as referring to a molecule (where the molecule is, for example, a polypeptide, a polynucleotide, or an antibody) that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also can be rendered substantially free of naturally associated components by isolation, using purification techniques well known in the art. Molecule purity or homogeneity may be assayed by a number of methods known in the art. For example, the purity of a polypeptide sample may be assayed using polyacrylamide gel electrophoresis and staining of the gel to visualize the polypeptide using techniques known in the art. For certain purposes, higher resolution may be provided by using HPLC or other means known in the art for purification.

As used herein, the terms “treating” or “treatment” or “to treat” or “alleviating” or “to alleviate” all refer to (1) therapeutic measures that cure, slow down, lessen symptoms of, and/or halt progression of a diagnosed pathologic condition or disorder and/or (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” according to the methods and molecules of the present disclosure if the patient shows, e.g., total, partial, or transient remission of a certain type of disorder.

As used herein, the term “substantially homogenous” means that the component can be at least about 90%, at least about 91%, at least about 92%, at least about 93%, at least about 94%, at least about 95%, at least about 96%, at least about 97%, at least about 98%, at least about 99%, and/or at least about 99.5% or higher than 99.5% of the final preparation or more.

The terms “polypeptide”, “peptide” and “protein” are used interchangeably herein to refer to chains of amino acids of any length. The chain may be linear or branched.

The term “subject” and “patient” are used interchangeably and refer to a living organism suffering from or prone to a condition that can be prevented or treated by administration of a molecule such as Pro-IFN alfa-2b, acetylated Pro-IFN alfa-2b, and/or mixture thereof as provided herein, and includes both humans and animals.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure. One example is E. Coli.

As used herein, any concentration range, percentage range, ratio range or integer range is to be understood to include the value of any integer as indicated per se, as well as within the recited range and, when appropriate, fractions thereof (such as one tenth and one hundredth of an integer), unless otherwise indicated.

As used herein, an “effective dosage”, “effective therapeutic amount”, or “effective amount” of drug, compound, or pharmaceutical composition is an amount sufficient to affect any one or more beneficial or desired results. In more specific aspects, an effective amount prevents, alleviates, ameliorates symptoms of disease, and/or prolongs the survival of the subject being treated. For prophylactic use, beneficial or desired results include, but not limited to eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include, but not limited to clinical results such as reducing one or more symptoms of a disease such as cancer. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” or “effective amount” may be considered in the context of administering one or more therapeutic agents, and a single agent may be considered to be given in an effective amount if, in conjunction with one or more other agents, a desirable result may be or is achieved.

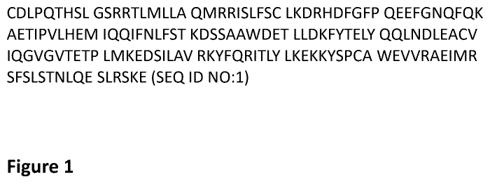

As used herein, the term “hIFN alpha 2b” or “hIFN-A2b” refer to the human version of interferon having category alpha in class 2b, wherein the amino acid sequence is in FIG. 1 (SEQ ID NO: 1).

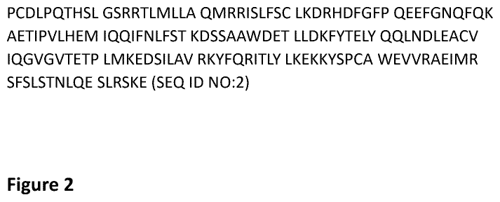

As used herein, the term “Pro-hIFN” or “Pro-hIFN-A2b” refer to modified hIFN alpha 2b having the amino acid sequence denoted in FIG. 2 (SEQ ID NO:2).

As used herein, the term “Acetylated Pro-hIFN” or “Acetylated Pro-hIFN-A2b” refer to modified hIFN alpha 2b having one or more amino acid acetylated at one or more positions of the sequence denoted in SEQ ID NO:2. The one or more acetylated positions may be selected from Proline at position 1 at the N-terminus, Lysine at position 50, Serine at position 116, and Serine at position 137.

As used herein, the term “per dose”, “dosage”, or “dose” means administering a given numeric amount of drug to a subject. Per dose can be administered in separate injections at about the same time and/or different time, so long as a subject receives the drug amount.

As used herein, the term “per injection” means administering the entire numeric amount of drug to a subject for a given dose in a single injection.

As used herein, “substantially” or “substantially all” means more than about 50%, 60%, 70%, 80%, 90%, 92.5%, 95%, 97.5%, 98%, 99%, 99.5%, 99.95% or 100% of a molecule.

As used herein, “vector” or “expression vector” means a construct, which is capable of delivering, and expressing one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “pharmaceutically acceptable carrier” or “pharmaceutical acceptable excipient” includes any material which, when combined with an active ingredient, allows the ingredient to retain biological activity or stability and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by known methods.

The term “intradermal administration”, “i.d.”, or “administered intradermally,” in the context of administering a substance to a mammal including a human, refers to the delivery of the substance into the dermis layer of the skin of the mammal. The skin of a mammal is composed of an epidermis layer, a dermis layer, and a subcutaneous layer. The epidermis is the outer layer of the skin. The dermis, which is the middle layer of the skin, contains nerve endings, sweat glands and oil (sebaceous) glands, hair follicles, and blood vessels. The subcutaneous layer is made up of fat and connective tissue that houses larger blood vessels and nerves. In contrast in intradermal administration, “s.c.”, or “subcutaneous administration”, refers to the administration of a substance into the subcutaneous layer and “topical administration” refers to the administration of a substance onto the surface of the skin.

As used herein, “about” mean within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 5%, 7.5%, 10%, 12.5%, 15%, 17.55, 20%, 22.5% 25%, 27.5%, 30%, 32.5%, 35%, 37.5%, or 40% of difference in either direction (positive or negative) compared to a reference value. Furthermore, particularly with respect to biological systems or processes, the terms can mean up to an order of magnitude or up to 1, 2, 3, 4, or 5-folds of a value. When particular values are provided in the application and claims, unless otherwise stated, the meaning of “about” should be assumed to be within an acceptable error range for that particular value. Reference to “about” a value or parameter herein includes (and describes) embodiments that are directed to that value or parameter per se. For example, description referring to “about X” includes description of “X”. Numeric ranges are inclusive of the numbers defining the range.

Peptide/Nucleotide Sequence of hIFN Alpha 2b and Pro-hIFN

In an embodiment, the present disclosure includes a biologics molecule having 165 amino acids, and one particular example is denoted in FIG. 1 as SEQ ID No.: 1. The biologics molecule is known as IFN alfa-2b.

In another embodiment, the present disclosure includes a biologics molecule having 166 amino acids Pro-IFN alfa-2b as shown in FIG. 2. In particular, it is bacterially produced with extra N-terminal Methionine (Met) and Proline (Pro) and the Met is removed via method known in the art.

Alternatively, or additionally, in another embodiment, one or more amino acid of Pro-IFN alfa-2b can be acetylated forming acetylated Pro-IFN alfa-2b, and/or mixture thereof. The one or more acetylated positions may be selected from Proline at position 1 at the N-terminus, Lysine at position 50, Serine at position 116, Serine at position 137, acetylated lysine at position 32, acetylated lysine at position 71, acetylated lysine at position 113, acetylated lysine at position 132, acetylated lysine at position 135 and/or acetylated tyrosine at position 136. Additionally, or alternatively, the mere fact that the detection of existence of one or more acetylated amino acid as disclosed, or in any amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product.

In another embodiment, two or more amino acids of Pro-IFN alfa-2b can be acetylated forming acetylated Pro-IFN alfa-2b, and/or mixture thereof. The two or more acetylated position may be at two or more selected from Proline at position 1 at the N-terminus, Lysine at position 50, Serine at position 116, Serine at position 137, acetylated lysine at position 32, acetylated lysine at position 71, acetylated lysine at position 113, acetylated lysine at position 132, and/or acetylated lysine at position 135, and/or acetylated tyrosine at position 136. For example, quantitative ratio of the two or more acetylated position may be at Proline at position 1 and Lysine at position 50; Proline at position 1 and Serine at position 116; Proline at position 1 and Serine at position 137; Lysine at position 50 and Serine at position 116; Lysine at position 50 and Serine at position 137; Serine at position 116 and Serine at position 137 or any combination thereof with an overall quantitative relative ratio of each can be 1:1 or 2:1 or 1:2. Additionally, or alternatively, the mere fact that the detection of existence of one or more acetylated amino acid as disclosed, or in any amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product.

In another embodiment, three or more amino acids of Pro-IFN alfa-2b can be acetylated forming acetylated Pro-IFN alfa-2b, and/or mixture thereof. The acetylated position may be at three or more selected from Proline at position 1 at the N-terminus, Lysine at position 50, Serine at position 116, Serine at position 137, lysine at position 32, lysine at position 113, lysine at position 132, and/or lysine at position 135. For example, the acetylated position may be at Proline at position 1, Lysine at position 50 and Serine at position 116; Proline at position 1, Lysine at position 50 and Serine at position 137; Lysine at position 50, Serine at position 116 and Serine at position 137 or any combination thereof with an overall quantitative relative ratio of 1:1:1, 2:1:1, 1:2:1, or 1:1:2.

In another embodiment, acetylated Pro-IFN alfa-2b may have at least four acetylated positions including Proline at position 1 at the N-terminus, Lysine at position 50, Serine at position 116, and Serine at position 137. Generally, the overall quantitative relative ratio of acetylated positions may be 1-5:1-5:1-5:1-5. Other nonlimiting examples of overall quantitative relative ratio of acetylated positions may include 1:1:1:1, 2:1:1:1, 1:2:1:1, 1:1:2:1, 1:1:1:2, 5:1:1:1, 1:5:1:1, 1:1:5:1, 1:1:1:5, 2:1:1:5, 1:2:1:5, or 1:1:2:5. In an embodiment, acetylated lysine at position 32, acetylated lysine at position 113, acetylated lysine at position 132, and/or acetylated lysine at position 135 were detected. The quantitative ratio of acetylated Lys32:Lys113:Lys132:Lys135 is about 12:1:7.8:3. In an embodiment, acetylated lysine at position 32, acetylated lysine at position 113, acetylated lysine at position 132, and/or acetylated lysine at position 135 were detected. The quantitative ratio of acetylated Lys32:Lys113:Lys132:Lys135 is about 4:03:2.6:1. In another embodiment, the acetylated position of Pro-hIFN alpha 2b are located at lysing at position 32, lysine at position 113, lysine at position 132, and/or lysine at position 135. In an embodiment, the overall quantitative relative ratio amount of lysing at position 32, lysine at position 113, lysine at position 132, and lysine at position 135 is about 12:1:7.8:3. In an embodiment, the acetylated position of Pro-hIFN alpha 2b located at lysine at position 50 (Lys50), acetylated lysine at position 71 (Lys 71), acetylated lysine at position 113 (Lys 113), and acetylated tyrosine at position 136 (Tyr 136) is about 32:1:91:28. Alternative, or additionally, in aspect, the quantitative ratio of the acetylated position of Pro-hIFN alpha 2b located at lysine at position 50 (Lys50), acetylated lysine at position 113 (Lys 113), and acetylated tyrosine at position 136 (Tyr 136) is about 1.14:3.25:1. Yet in an embodiment, the quantitative ratio of Pro-hIFN alpha 2b located at lysine at position 71 (Lys71) and acetylated tyrosine at position 136 (Tyr 136) is about 1:28. Additionally, or alternatively, the mere fact that the detection of existence of one or more acetylated amino acid as disclosed, or in any amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product. The nucleotide sequences encoding such amino acid can vary such as using codon optimization, if needed, and depending on which host cell is used. Such method is known in the art. The nucleotide sequence can be cloned into a vector and transfect into a bacterial host cell for protein expression by methods known in the art.

Additionally, or alternatively, the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, or other amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product.

In an embodiment, acetylated lysine at position 32, acetylated lysine at position 113, acetylated lysine at position 132, and/or acetylated lysine at position 135 were detected. The ratio of acetylated Lys32:Lys113:Lys132:Lys135 is about 4:03:2.6:1. Additionally, or alternatively, the mere fact that the detection of existence of one or more acetylated amino acid as disclosed, or in any amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product.

ii. Method of Making

Previously, a method to generate Pro-IFN A2b is known. See U.S. Pat. No. 8,106,160, the content of which are incorporated herein by reference in their entirety. In U.S. Pat. No. 8,106,160, no acetylated Pro-IFN A2b was reported. Here, in an embodiment, the method of making is not identical and acetylated Pro-hIFN alpha 2b can be produced, but not necessarily all the time. In an embodiment, the presence or occurrence of acetylated Pro-hIFN alpha 2b can be detected at a frequency of about 90%, 91%, 92%, 93%, 94%, 95%, 96%, 97%, 98%, 99%, 99.5%, 99.9%, 99.99% and/or higher 99.99% over time.

In an embodiment, the present disclosure provides new methods of generating and making Acetylated Pro-hIFN A2b, Pro-hIFN A2b, and/or mixture thereof.

Briefly, and in general, DNA encoding amino acid of SEQ ID No:2 are constructed in a vector, and such vectors encoding Pro-hIFN are transfected to host cells such as E. Coli. Then, the host cells are first inoculated, and cultured in a fermentor. Induction is conducted with IPTG (Isopropyl β-D-1-thiogalactopyranoside) to induce protein product in the host cell, and then the cells are harvested. The cell pellet is then stored. Then, the cell pellet is homogenized to obtain a crude lysate which contains the protein Pro-hIFN. The crude lysate protein is then refolded by method known in the art, and clarified to obtain crude refolded protein solution. The crude solution is then concentrated and subjected to filtration, chromatography, and/or hydrophobic interaction chromatography to obtain purified Acetylated Pro-hIFN A2b, Pro-hIFN A2b, and/or mixture thereof. Additionally, or alternatively, the mere fact that the detection of existence of one or more acetylated amino acid as disclosed, or in any amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product.

In another embodiment, additionally, or alternatively, some of the fermentation parameters can be adjusted to improve yield, as disclosed in United States Patent Application number US20250101484A1. The contents of which are incorporated herein by reference in its entirety. For example, part of the fermentation can be performed including the steps of: carrying out seeding (time S0) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I0 (time at initiation of induction) or I1 or both; optionally, supplying a nitrogen source to any of the one or more fermenters, individually, at one or more time points selected from about S5, S6, S7, S8, S9 and S10. In an embodiment, said DO level exceeds above about 35% to 40% and said agitation rate exceeds about 300 to 700 rpm. In another embodiment, said agitation rate exceeds about 500 to 600 rpm and said pH exceeds about 7.0 to 7.4. Yet in another embodiment, the nitrogen source is supplied, individually, at about I0 or I1 or both, and at one or more time points selected from about S5, S6, S7, S8, S9 and S10. In an embodiment, a carbon source feeding solution is supplied at least once to at least one fermenter during the recombinant protein fermentation, wherein the carbon source feeding solution is a glucose feeding solution, fructose feeding solution, galactose feeding solution, pyruvate feeding solution, or any combination thereof. In another embodiment, a cumulative total of about 800 to 1500 g of glucose is supplied to at least one fermenter, per about 5 L fermenter capacity, by the end of the recombinant protein fermentation. Yet in another embodiment, the nitrogen source supplied to the one or more fermenters, individually, comprises potassium. In another embodiment, the nitrogen source supplied to the one or more fermenters, individually, comprises a yeast extract, peptone, soytone, urea, tryptone, or any combinations thereof. Yet in an embodiment, about 40 to 150 mL of a yeast extract solution is supplied the one or more fermenters each time, individually; optionally about 300 to 1500 mL of a yeast extract solution is supplied to the one or more fermenters each time, individually. In another embodiment, wherein the recombinant protein fermentation is performed with an inoculum ratio of about 6.0 to 8.5%. In an embodiment, said prokaryote host cell comprises an E. coli BLR-Codon Plus (DE3) cell. In another embedment, the fermentation process further comprising a step of collecting the culture of prokaryote host cell or a sample of the culture in the one or more fermenters after induction of expression of the recombinant protein, individually. In another embodiment, the fermentation process further comprising processing inclusion bodies in the collected culture of prokaryote cell or sample to obtain a crude protein extract. Yet in another embodiment, the culture or sample are collected after or at about I4 to I8. In an embodiment, the fermentation process the expressed recombinant protein in the crude protein extract, wherein the yield is about 5 to 10 g of the recombinant protein in a fermenter having about a 5 L capacity; optionally, wherein the yield is about 20 to 30 g of the recombinant protein in a fermenter having about a 40 L capacity. In an embodiment, not all, and in limited occasion, no acetylated Pro-hIFN A2b are produced. Additionally, or alternatively, the mere fact that the detection of existence of one or more acetylated amino acid as disclosed, or in any amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product.

Additionally, or alternatively, in the case that acetylation do occur, the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, or other amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any manufacturing step(s) of any part of the present disclosure's method was utilized to manufacture Pro-hIFN A2b intermediate and/or the final product.

In an embodiment, the acetylation of Pro-hIFN alpha 2b can be induced by adding Acetic anhydride in the amount of between about 0.5-1.5 of Pro-hIFN alpha 2b: between about 0.05-0.15 Acetic anhydride in molar ratio. In an aspect, the molar ratio can be between about 0.9-1.1 of Pro-hIFN alpha 2b: between about 0.10-0.12 Acetic anhydride in molar ratio. Additionally, or alternatively, the mere fact that the detection of existence of one or more acetylated amino acid as disclosed, or in any amount or combinations thereof, indicates the present one or more manufacturing procedure step was used to manufacture Pro-hIFN A2b intermediate and/or the final product. In another embodiment, the detection of the presence of acetylated amino acids, and/or overall quantitative any ratio before, during, or after any part of the present disclosure's manufacturing step(s) was utilized to manufacture Pro-hIFN A2b intermediate and/or the final product.

### Pharmaceutically Acceptable Carriers

The composition used in the present disclosure, whether Acetylated Pro-hIFN A2b, Pro-hIFN A2b, and/or mixture thereof alone, or with any combinations of other active ingredients thereof, can further comprise pharmaceutically acceptable carriers, excipients, or stabilizers, in the form of lyophilized formulations or aqueous solutions. Carriers, excipients, or stabilizers can be nontoxic to recipients at the dosages and concentrations, and can comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN (trademarked), PLURONICS (trademarked).

### Dosage

In an embodiment, a subject can be treated with Pro-IFN alfa-2b, acetylated Pro-IFN alfa-2b, and/or mixture thereof for a disease. In another embodiment, a disease or indication can be myelofibrosis of any subtype (e.g., idiopathic, primary, and/or early), polycythemia vera (high or low risk), essential thrombocythaemia, and/or chronic myeloid leukemia.

Yet in an embodiment, a treatment period using Pro-IFN alfa-2b, acetylated Pro-IFN alfa-2b, and/or mixture thereof can be at least about 1, 2, 3, 4, 5, 6, 7, 8, 9, 10, 11, 12, 24, 36, 42, 48, 54, 60, 66, 72, 78, 84 or more than 84 months. In some embodiments, the treatment period is about 0.1, 0.25, 0.5, 0.75, 1, 2, 3, 4, 4.5, 5, 5.5, 6, 6.5, 7, 7.5, 8, 8.5, 9, 9.5, 10 or more than 10 years. In some embodiments, the treatment period is at least about 0.1, 0.25, 0.5, 0.75, 1, 2, 3, 4, 5, 6, 7, 8, 12, 14, 15, 16, 17, 18, 19, 20, 24, 28, 32, 36, 40, 44, 48, 52, 56, 60 weeks or longer than 60 weeks.

In another embodiment, Pro-IFN alfa-2b, acetylated Pro-IFN alfa-2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes. Alternatively, or additionally, a dose or injection of Pro-IFN alfa-2b, acetylated Pro-IFN alfa-2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg. Alternatively, or additionally, a dose or injection of 135 μg or 180 μg of Pro-IFN alfa-2b, acetylated Pro-IFN alfa-2b, and/or mixture thereof can be administered.

In another embodiment, Pro-IFN alfa-2b, acetylated Pro-IFN alfa-2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every week. Alternatively, or additionally, a dose or injection of Pro-IFN alfa-2b, acetylated Pro-IFN alfa-2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every week. Alternatively, or additionally, a dose or injection of 135 μg or 180 μg of Pro-IFN alfa-2b, acetylated Pro-IFN alfa-2b, and/or mixture thereof can be administered every week.

In another embodiment, Pro-IFN alfa-2b, acetylated Pro-IFN alfa-2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every 2 weeks. Alternatively, or additionally, a dose or injection of Pro-IFN alfa-2b, acetylated Pro-IFN alfa-2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every 2 weeks. Alternatively, or additionally, a dose or injection of 135 μg or 180 μg of Pro-IFN alfa-2b, acetylated Pro-IFN alfa-2b, and/or mixture thereof can be administered every 2 weeks.

Yet in another embodiment, Pro-IFN alfa-2b, acetylated Pro-IFN alfa-2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every 3 weeks. Alternatively, or additionally, a dose or injection of Pro-IFN alfa-2b, acetylated Pro-IFN alfa-2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every 3 weeks. Alternatively, or additionally, a dose or injection of 135 μg or 180 μg of Pro-IFN alfa-2b, acetylated Pro-IFN alfa-2b, and/or mixture thereof can be administered every 3 weeks.

In another embodiment, Pro-IFN alfa-2b, acetylated Pro-IFN alfa-2b, and/or mixture thereof can be administered for treating myelofibrosis of any subtype (e.g., idiopathic, primary, and/or early), polycythemia vera (high or low risk), essential thrombocythaemia, and/or chronic myeloid leukemia using between about 50 to 540 μg per dose or per injection subcutaneously, via i.d., or other routes every 4 weeks. Alternatively, or additionally, a dose or injection of Pro-IFN alfa-2b, acetylated Pro-IFN alfa-2b, and/or mixture thereof can be administered during the treatment period ranges from about 250 to about 650 μg. The dose/injection can also be up to about 250 μg, up to about 255 μg, up to about 260 μg, up to about 265 μg, up to about 270 μg, up to about 275 μg, up to about 280 μg, up to about 285 μg, up to about 290 μg, up to about 295 μg, up to about 300 μg, up to about 305 μg, up to about 310 μg, up to about 315 μg, up to about 320 μg, up to about 325 μg, up to about 330 μg, up to about 335 μg, up to about 340 μg, up to about 345 μg, up to about 350 μg, up to about 400 μg, up to about 450 μg, up to about 500 μg, up to about 540 μg, or up to about 650 μg every 4 weeks. Alternatively, or additionally, a dose or injection of 135 μg or 180 μg of Pro-IFN alfa-2b, acetylated Pro-IFN alfa-2b, and/or mixture thereof can be administered every 4 weeks.

In one embodiment, Pro-IFN alfa-2b, acetylated Pro-IFN alfa-2b, and/or mixture thereof can be administered or dosed for treating myelofibrosis of any subtype (e.g., idiopathic, primary, and/or early), polycythemia vera (high or low risk), essential thrombocythaemia, and/or chronic myeloid leukemia according to the formula 0.75-1.5 μg/kg/wk where the μg is amount of Pro-IFN alfa-2b, acetylated Pro-IFN alfa-2b, and/or mixture thereof, kg is a subject's weight and wk is week.

In some embodiments, an initial (starting) dose for treating myelofibrosis of any subtype (e.g., idiopathic, primary, and/or early), polycythemia vera (high or low risk), essential thrombocythaemia, and/or chronic myeloid leukemia can be about 250 to about 500 μg (e.g., about 250 μg, about 300 μg, about 350, about 400 μg, about 450 μg, or about 500 μg) of Pro-IFN alfa-2b, acetylated Pro-IFN alfa-2b, and/or mixture thereof administered to the subject. The initial dose/injection can be maintained or varied during the treatment period depending on patient's need and/or physician's recommendation.

In any of the methods or treatment periods described herein, Pro-IFN alfa-2b, acetylated Pro-IFN alfa-2b, and/or mixture thereof can be titrated. As non-limiting example, a subject can be treated for treating myelofibrosis of any subtype (e.g., idiopathic, primary, and/or early), polycythemia vera (high or low risk), essential thrombocythaemia, and/or chronic myeloid leukemia with a lower starting dose/injection (e.g., about 50 μg, about 100 μg, about 150 μg, about 200 μg, or about 250 to about 500 μg) of the Pro-IFN alfa-2b, acetylated Pro-IFN alfa-2b, and/or mixture thereof. If the subject responds well (e.g., lack of significant drug-related adverse events, significant self-reported discomfort, abnormal hematological responses, or other symptoms) after a time (e.g., between about 1 to 26 weeks, 1 to 52 weeks, or more than 52 weeks), the dose/injection given to the subject can be increased incrementally (e.g., by between about 50 to 250 μg, such as at about 50 μg, about 75 μg, about 100 μg, about 125 μg, about 150 μg, about 200 μg, about 250 μg or a combination thereof) every 2 to 16 weeks (e.g., every 1, 2, 3, 4, 5, 6, 7, 8, 9, 10, 11, 12, 13, 14, 15, or 16 weeks, or a combination thereof) until the dose reaches a target dose (e.g., at least about 400 μg, at least about 425 μg, at least about 450 μg, at least about 475 μg, at least about 500 μg, at least about 525 μg, at least about 550 μg, or at least about 650 μg). After that, a target dose can be maintained during the treatment period, increased, and/or decreased depend on subject's condition. The dose/injection can also be increased successively until the desired target dose is reached. For example, if the Pro-IFN alfa-2b, acetylated Pro-IFN alfa-2b, and/or mixture thereof can be administered once every 1, 2, 3, 4, 5, 6, 7, or 8 weeks, the dose/injection can be increased every 1, 2, 3, 4, 5, 6, 7 or 8 weeks, respectively. In some embodiments, a subject can be given a starting dose/injection of 250 μg (i.e., at week 0). If the subject responds well to the initial dose/injection, the dose/injection can be increased by about 100 to about 150 μg every 2 to 8 weeks until it reaches a target dose of about 500 μg, about 550 μg, and/or 600 μg. For example, a 250-350-500 μg dosing schedule can be implemented (i.e., about 250 μg at week 0, about 350 μg between about week 2 to 8, and about 500 μg at the third administration 2 to 8 weeks after the initial second dose, without other intervening doses). Alternative, a subject can be for treating myelofibrosis of any subtype (e.g., idiopathic, primary, and/or early), polycythemia vera (high or low risk), essential thrombocythaemia, and/or chronic myeloid leukemia by given a starting dose/injection of about 350 μg and a second dose of about 500 μg between about 2 to 16 weeks thereafter without an intervening dose (i.e., 350-500). Exemplary dosing schedules can be abbreviated, nonetheless, each number is approximated. For example, 250-350-500 includes 1st dose/injection administered at about 250 μg, 2nd dose/injection administered at about 350 μg, and 3rd dose/injection administered at about 500 μg. Other embodiments include, but are not limited to: 250-400-500, 250-400, 250-500, 250-400-500, 250-450, 250-350-400-500, 250-300-400-500, 250-350-450-500, 250-350-450, 250-250-350-500, 250-250-250-350-500, 250-350-350-500, 250-500-500-500 (and remain at 500 afterwards), 350-500, 350-400-500, 350-400-450-500, 350-450-500, 350-400, 350-450, 350-350-500, 350-350-350-500, 400-450-500, 400-500, 400-400-500, 450-500 μg. In some embodiments, the target dose and/or desirable effect is reached between about 1 to 13 weeks, about 1 to 26 weeks, 1 to 52 weeks, or more than 52 weeks from the initial administration. During the titration process, any dose, prior to reaching the target dose, may be maintained for a time period (e.g., between about 4 to 16 weeks) or a number of successive doses dose/injection (e.g., between 2 to 8 successive doses dose/injection, e.g., 250-350-350-500 μg) or reduced depending on the subject's response. In some embodiments, the target dose is reached within about 2 to 8 successive doses. In further embodiments, once the subject is clinically stable, the dose/injection can be maintained at a constant level for a given treatment period, which can be at least about 1, 2, 3, 4, 5, 6, 7, 8, 12, 14, 15, 16, 17, 18, 19, 20, 24, 28, 32, 36, 40, 44, 48, 52, 56, 60 weeks or longer.

In an aspect, an initial dose/injection or starting dose/injection of Pro-IFN alfa-2b, acetylated Pro-IFN alfa-2b, and/or mixture thereof refers to the first dose administered to a subject for treating myelofibrosis of any subtype (e.g., idiopathic, primary, and/or early), polycythemia vera (high or low risk), essential thrombocythaemia, and/or chronic myeloid leukemia during a treatment period (i.e., week 0), wherein, prior to the treatment period, the subject is interferon-treatment naïve or has not been administered the same active ingredient as Pro-IFN alfa-2b, acetylated Pro-IFN alfa-2b, and/or mixture thereof. A subject who is interferon-treatment naïve is a subject who has not been treated with any form of interferon, whether pegylated or non-pegylated.

In another embodiment, all of the above recited dosages for treating myelofibrosis of any subtype (e.g., idiopathic, primary, and/or early), polycythemia vera (high or low risk), essential thrombocythaemia, and/or chronic myeloid leukemia can be administered per dose, and/or per injection. Non-limiting example include administering about 250-350-500 μg of drug using about 50 μg per about 5 injections at about the same time and/or different times, so long as the subject receives a total of about 250 μg dose, or about 100 μg per injection about 5 times at about the same time and/or different times for a total of about 500 μg dose. The combination can be picked and choose depending on patient or subject convenience and/or medical need. In another non-limiting example, the entire about 250 μg can be all administered in a single injection (per injection) at a single time point for the desired 250 μg dose.

In an embodiment, any of the above-mentioned dosage, or dosage scheme can be use, and/or in a mix-and-match fashion for treating myelofibrosis of any subtype (e.g., idiopathic, primary, and/or early), polycythemia vera (high or low risk), essential thrombocythaemia, and/or chronic myeloid leukemia, depending on the subject's tolerance and a physician's assessment (e.g., based on number and/or types of AE). In another embodiment, all of the above recited dosages can be administered per dose, and/or per injection. Non-limiting example include administering about 250 μg, 350 μg of drug using about 600 μg per about 3 injections at about the same time and/or different times, so long as the subject receives a total of about 600 μg dose. The combination can be picked and choose depending on patient or subject convenience and/or medical need. In another non-limiting example, the entire about 6 mg can be all administered in a single injection (per injection) at a single time point.

### Administration Methods

In an aspect, Pro-IFN alfa-2b, acetylated Pro-IFN alfa-2b, and/or mixture thereof can be used for subcutaneous injection to a subject. Alternative, or additionally, Pro-IFN alfa-2b, acetylated Pro-IFN alfa-2b, and/or mixture thereof can be administered for treating myelofibrosis of any subtype (e.g., idiopathic, primary, and/or early), polycythemia vera (high or low risk), essential thrombocythaemia, and/or chronic myeloid leukemia via topical contact, intravenous, intraperitoneal, intramuscular, intralesional, intranasal or the implantation of a slow-release device e.g., a mini-osmotic pump, to a subject. Other administration is by any route including parenteral, and transmucosal (e.g., oral, nasal, vaginal, rectal, or transdermal). Parenteral administration includes, e.g., intravenous, intramuscular, intra-arteriole, intradermal, intraperitoneal, intraventricular, and/or intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches.

In certain embodiments, Pro-IFN alfa-2b, acetylated Pro-IFN alfa-2b, and/or mixture thereof is administered for treating myelofibrosis of any subtype (e.g., idiopathic, primary, and/or early), polycythemia vera (high or low risk), essential thrombocythaemia, and/or chronic myeloid leukemia once daily, about once weekly, about once every two weeks, about once every three weeks, about once every four weeks, about once every five weeks, about once every six weeks, or about once every three months. Pro-IFN alfa-2b, acetylated Pro-IFN alfa-2b, and/or mixture thereof can be co-administered within about twenty-four hours of a dose of chemotherapy and/or radiation therapy.

In certain embodiments, Pro-IFN alfa-2b, acetylated Pro-IFN alfa-2b, and/or mixture thereof is co-administered at least once about 3, once about 7, once about 10, once about 14, once about 17 or once about 21 days before a dose of chemotherapy and/or radiation therapy. In an embodiment, Pro-IFN alfa-2b, acetylated Pro-IFN alfa-2b, and/or mixture thereof can be co-administered to a patient immediately after, at about the same time, and/or any time during chemo and/or radiation therapy.

### Uses

In one embodiment, Pro-hIFN alpha 2b, acetylated Pro-hIFN alpha 2b, an/or mixture thereof can be used to treat a subject with myelofibrosis of any subtype (e.g., idiopathic, primary, and/or early), polycythemia vera (high or low risk), essential thrombocythaemia, and/or chronic myeloid leukemia of any subtype.

Yet in another embodiment, the existence of acetylated Pro-hIFN A2b indicates that the any part of the present disclosure's method of making Pro-hIFN a2b was utilized. Non-limiting example include detecting the presence of the acetylated position of Pro-hIFN alpha 2b located at the Proline at position 1 and position 50 of Lysine. In an embodiment, the detection amount of quantitative relative ratio of Pro¹-acetylated Pro-hIFN alpha 2b to acetylated Lys⁵⁰ Pro-hIFN alpha 2b can be about 2:1 or 1:2. In another embodiment, the detection amount of quantitative relative ratio of Pro¹-acetylated Pro-hIFN alpha 2b to acetylated Lys⁵⁰ Pro-hIFN alpha 2b can be about 1:1. In one embodiment, acetylated lysine at position 32, acetylated lysine at position 113, acetylated lysine at position 132, and/or acetylated lysine at position 135 were detected. The ratio of acetylated Lys32:Lys113:Lys132:Lys135 can be about 12:1:7.8:3.

In another embodiment, the existence of acetylated Pro-hIFN A2b indicates that the any part of the present disclosure's method of making Pro-hIFN a2b was utilized. Non-limiting examples include detecting the presence of the acetylated position of Pro-hIFN alpha 2b located at the initial Proline at position 1, position 50 of Lysine, and position 116 of serine. In an embodiment, the detection amount of Pro¹-acetylated Pro-hIFN alpha 2b to acetylated Lys⁵⁰ Pro-hIFN alpha 2b to acetylated Serin¹¹⁶ Pro-hIFN alpha 2b can have a quantitative relative ratio of about 1:1:1. In an embodiment, the amount of Pro¹-acetylated Pro-hIFN alpha 2b to acetylated Lys⁵⁰ Pro-hIFN alpha 2b to acetylated Serin¹¹⁶ Pro-hIFN alpha 2b can have a quantitative relative ratio of about 2:1:1 or 1:1:2.

In an embodiment, the Pro-hIFN alpha 2b does not have acetylated position of located at the initial Proline at position 1.

In another embodiment, the existence of acetylated Pro-hIFN A2b indicates that the any one step and/or any part of the present disclosure's method of making Pro-hIFN a2b was utilized. Non-limiting examples include detecting the presence of the acetylated position of Pro-hIFN alpha 2b located at the initial Proline at position 1, position 50 of Lysine, position 116, and position 137 of serine, lysing at position 32, lysine at position 113, lysine at position 132, and/or lysine at position 135 at the cloning step, at the protein expression step, at the fermentation step, at the inclusion body step, at the gel electrophoresis step, at the purification step, and/or at the final product batch. In an embodiment, the detection amount of Pro¹-acetylated Pro-hIFN alpha 2b to acetylated Lys⁵⁰ Pro-hIFN alpha 2b to acetylated Serin¹¹⁶ Pro-hIFN alpha 2b to acetylated Serine¹³⁷ Pro-hIFN alpha 2b can have an quantitative relative ratio of about 1:1:1:5. In an embodiment, the detection amount of Pro¹-acetylated Pro-hIFN alpha 2b to acetylated Lys⁵⁰ Pro-hIFN alpha 2b to acetylated Serin¹¹⁶ Pro-hIFN alpha 2b to acetylated Serine¹³⁷ Pro-hIFN alpha 2b can have a quantitative relative ratio of about 2:1:1:5.

In another embodiment, the existence of acetylated Pro-hIFN A2b or in any quantitative amount or ratio thereof, indicates that the any one step and/or any part of the present disclosure's method of making Pro-hIFN a2b was utilized. Non-limiting examples include detecting the presence of the acetylated position of Pro-hIFN alpha 2b located at the initial Proline at position 1, position 50 of Lysine, position 116, and position 137 of serine, lysing at position 32, lysine at position 113, lysine at position 132, lysine at position 135, lysine at position 71, and/or tyrosine at position 136 at the cloning step, at the protein expression step, at the fermentation step, at the inclusion body step, at the gel electrophoresis step, at the purification step, and/or at the final product batch. In an embodiment, the detection amount of Pro¹-acetylated Pro-hIFN alpha 2b to acetylated Lys⁵⁰ Pro-hIFN alpha 2b to acetylated Serin¹¹⁶ Pro-hIFN alpha 2b to acetylated Serine¹³⁷ Pro-hIFN alpha 2b can have an quantitative relative ratio of about 1:1:1:5. In an embodiment, the detection amount of Pro¹-acetylated Pro-hIFN alpha 2b to acetylated Lys⁵⁰ Pro-hIFN alpha 2b to acetylated Serin¹¹⁶ Pro-hIFN alpha 2b to acetylated Serine¹³⁷ Pro-hIFN alpha 2b can have a quantitative relative ratio of about 2:1:1:5.

Surprising Advantages and/or Improvement

In an embodiment, the present disclosure describes surprising acetylation of amino acid in a prokaryotic manufacturing system (e.g., E. Coli) as compared to eukaryotic system. It is known in the art that N alpha-acetylation is almost exclusively restricted to eukaryotic structural proteins. Examples of N-terminal acetylation are rare in prokaryotic systems, but it is known that in prokaryotes, acetylation mostly occurs N-terminal, and for interferon, on the N terminal Cys residue of recombinant human interferon-2b produced in Escherichia coli as a favorite site for acetylation (See for example, Ahsana et al. Journal of Biotechnology. Volume 184, 20 Aug. 2014, Pages 11-16) For acetylation, it is a post-translational process, requiring the presence of the enzyme N alpha-acetyltransferase and the acetyl donor acetylcoenzyme A. Amino acids predominantly present at the N terminus of N alpha-acetylated proteins can be, for example, alanine, serine, and methionine. The occurrence of these residues is apparently a prime target for acetylation. However, none of the above are the case for the present disclosure. The Pro-hIFN alpha 2b has a proline to begin with followed by cysteine. In addition, the raw materials do not contain N alpha-acetyltransferase and the acetyl donor acetylcoenzyme A. Further, the acetylation of other amino acids of the present disclosure were not limited to N-terminal amino acid. Therefore, the presence of the acetylation of Pro-hIFN alpha 2b is surprising and unexpected.

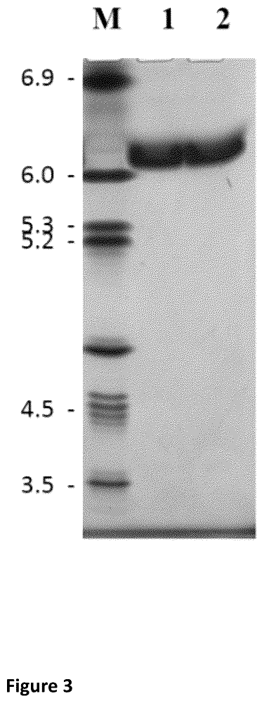

In addition, as compared to the previously reported method of making Pro-hIFN alpha 2b (see U.S. Pat. No. 8,106,160, the contents are all incorporated herein in their entirety), the present disclosure's method production of Pro-hIFN alpha 2b with and/or without acetylated Pro-hIFN alpha 2b can be used as an indicator that any part of the present disclosure's method of making was utilized and/or performed. For example, see FIG. 3 of U.S. Pat. No. 8,106,160, and/or the lack of known acetylated Pro-hIFN alpha 2b.

Further, in an embodiment acetylated Pro-hIFN alpha 2b does not always present in a given batch of manufacturing. In an occasion, acetylated Pro-hIFN alpha 2b was not detected. In an embodiment, the presence or occurrence of acetylated Pro-hIFN alpha 2b can be detected at a frequency of 90%, 91%, 92%, 93%, 94%, 95%, 96%, 97%, 98%, 99%, 99.5%, 99.9% and/or 99.99% over time.

Yet in another embodiment, the acetylated positions of Pro-hIFN alpha 2b are located at the initial Proline at position 1 of the N-terminus and position 50 of Lysine. In an embodiment, the overall quantitative relative ratio amount of Pro¹-acetylated Pro-hIFN alpha 2b to acetylated Lys⁵⁰ Pro-hIFN alpha 2b is about 2:1 or 1:2. In another embodiment, the overall relative ratio amount of Pro¹-acetylated Pro-hIFN alpha 2b to acetylated Lys⁵⁰ Pro-hIFN alpha 2b is about 1:1.

In another embodiment, the acetylated positions of Pro-hIFN alpha 2b are located at the initial Proline at position 1, position 50 of Lysine, and position 116 of serine.

In an embodiment, the overall quantitative relative ratio amount of Pro¹-acetylated Pro-hIFN alpha 2b to acetylated Lys⁵⁰ Pro-hIFN alpha 2b to acetylated Serin¹¹⁶ Pro-hIFN alpha 2b is about 1:1:1. In an embodiment, the overall quantitative relative ratio amount of Pro¹-acetylated Pro-hIFN alpha 2b to acetylated Lys⁵⁰ Pro-hIFN alpha 2b to acetylated Serin¹¹⁶ Pro-hIFN alpha 2b is about 2:1:1.

In another embodiment, the acetylated position of Pro-hIFN alpha 2b are located at the initial Proline at position 1, position 50 of Lysine, position 116, and position 137 of serine. In an embodiment, the overall quantitative relative ratio amount of Pro¹-acetylated Pro-hIFN alpha 2b to acetylated Lys⁵⁰ Pro-hIFN alpha 2b to acetylated Serin¹¹⁶ Pro-hIFN alpha 2b to acetylated Serine¹³⁷ Pro-hIFN alpha 2b is about 1:1:1:5. In an embodiment, the overall quantitative relative ratio amount of Pro¹-acetylated Pro-hIFN alpha 2b to acetylated Lys⁵⁰ Pro-hIFN alpha 2b to acetylated Serin¹¹⁶ Pro-hIFN alpha 2b to acetylated Serine¹³⁷ Pro-hIFN alpha 2b is about 2:1:1:5.

In another embodiment, the acetylated position of Pro-hIFN alpha 2b are located at lysing at position 32, lysine at position 113, lysine at position 132, and/or lysine at position 135. In an embodiment, the overall quantitative relative ratio amount of lysing at position 32, lysine at position 113, lysine at position 132, and lysine at position 135 is about 12:1:7.8:3.

### EXAMPLES

### Example 1

Preparation of Recombinant N-Terminal Modified hIFN-α2b (Pro-hIFN alpha 2b, acetylated Pro-hIFN alpha 2b, an/or mixture thereof)

This method is not identical to known method of making Pro-hIFN A2b. Here, a nucleic acid encoding Pro-hIFN-α2b was obtained by amplifying hIFN-α2b using primers. Briefly, codens, ATG and CCG (encoding Met and Pro, respectively) were added to the 5′ of coden that encodes Cys of mature hIFN-α2b. This nucleic acid was cloned into expression vector pET-24a.

pET-24a vectors, carrying the Pro-hIFN-α2b genes, were then transformed into E. coli BLR-(DE 3)-RIL strain. E. coli clones expressing high levels of the protein were selected. The nascent protein expressed from the Pro-hIFN-α2b gene has a Met at its N-terminus (Met-Pro-Cys-). This Met residue was removed in E. coli via internal enzymatic digestion, resulting in a mature protein having an N-terminal Pro, which is linked to the Cys1 of hIFN-α2b (Pro-Cys-).

The E. coli clone which will express Pro-hIFN-α2b was cultured in a 1000 ml flask containing 250 ml SYN Broth medium (soytone, yeast extract, and NaCl) with kanamycin (50 μg/mL) and chloramphenicol (50 μg/mL) at about 37° C., rotate at 200 rpm for about between 14 to16 hours overnight. About 220 ml of the overnight culture were then transferred to a 5-liter jar fermentor containing 3 L basic medium (10 g/L glucose, 0.7 g/L MgSO₄·7H₂O, 4 g/L (NH₄)₂HPO₄, 3 g/L KH₂PO₄, 6 g/L K₂HPO₄, 1.7 g/L citrate, 10 g/L yeast extract, and 2 g/L isoleucine) with kanamycin (25 μg/mL), chloramphenicol (25 μg/mL), and between about 0.5% to 1% (v/v) trace elements (10 g/L of FeSO4.7H₂O, 2.25 g/L of ZnSO₄·7H₂O, 1 g/L of CuSO₄·5H₂O, 0.5 g/L of MnSO₄·H₂O, 0.3 g/L of H₃BO₃, 2 g/L of CaCl₂·2H₂O, 0.1 g/L of (NH₄)₆Mo₇O₂₄, between about 0.81 to 0.84 g/L EDTA, and 50 ml/L HCl). The dissolved oxygen concentration in the medium was controlled at between about 40%, or above 40% to 45% and its pH maintained at between about 6.9-7.1 by adding between about 25% to 37% ammonia water whenever necessary. A feeding solution containing between about 600-800 g/L of glucose and about 20 g/L of MgSO₄·7H₂O was prepared. When the dissolved oxygen rose to a value greater than the set point, an appropriate volume of the feeding solution was added to increase the glucose concentration in the culture medium. The expression of the Pro-hIFN-α2b gene was induced by IPTG at a final concentration of between about 0.6-0.8 mM, then addition of feeding material (yeast extract and trace element). E. coli cells expressing these proteins were collected post five hours after IPTG induction.

The collected E. coli cells were resuspended in TEN buffer (50 mM Tris-HCl, pH 7.0; 1 mM EDTA, and 100 mM NaCl) in a ratio of between about 1:3 to 1:10 (wet weight g/mL), disrupted by a homogenizer, and then centrifuged at about 10,000 rpm for about 20 min. The pellet containing inclusion bodies (IBs) was washed twice with TEN buffer and centrifuged as described above, suspended in a ratio of 1 ml solution: about 2.0-2.5 g pellet wet weight g/mL of a between about 3-4 M guanidium HCl (GnHCl) aqueous solution, and then centrifuged at between about 5,000 to 10,000 rpm for between about 15 to 40 min. The IBs, containing recombinant hIFN-α2b, were then solubilized in a ratio of about 0.8 to 1 ml solution: between about 1.8-2.7 g pellet wet weight g/mL in 50 mL of between about 5-6 M GuHCl with about 5 mM DTT, which was then stirred at room temperature for about 1.5 hr followed by centrifugation at between about 10,000 to 20,000 rpm for between about 20 to 40 min at about 25° C. The supernatant was collected. In this process, the recombinant Pro-hIFN A2b (and/or acetylated Pro-hIFN A2b) protein was denatured.

At this stage the mere fact that the detection of existence of one or more acetylated amino acid from Proline at position 1 at the N-terminus, Lysine at position 50, Serine at position 116, Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b

Multiple runs were performed using procedures disclosed in the present application, and not all runs would produce acetylated Pro-hIFN A2b.

Additionally, or alternatively, in one of the multiple run, acetic anhydride can be added in the about of about 1:about 0.11 molar ratio of Pro-hIFN A2b: Acetic anhydride.

### Example 2

### Gel Electrophoresis

The protein concentration of the crude lysate was determined by BCA protein assay, and the Pro-hIFN A2b (and/or acetylated Pro-hIFN A2b) was determined by isoelectric focusing gel electrophoresis. The charge distribution of Pro-IFN alfa-2b (and/or acetylated Pro-hIFN A2b) was also evaluated by isoelectric focusing (IEF) gel electrophoresis. The results are illustrated in FIG. 3, with duplicated lanes each loaded with about 15 μg with a marker line loaded with about 10 μL on the very left labelled as “M”. FIG. 3 shows an IEF profile that gives major band with the isoelectric point (pI) of 6.1 for Pro-IFN alfa-2b. Results are consistent with the detection of an IEF main band at pI 6.1±0.1. Currently known IEF method is not as sensitive enough to detect acetylated Pro-hIFN alpha 2b.

At this stage the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135, and/or acetylated tyrosine at position 136, in any other quantitative amount, ratio, or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the any part of the present disclosure's method was utilized to manufacture Pro-hIFN A2b.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b

### Example 3

Refolding of Pro-hIFN alpha 2b, acetylated Pro-hIFN alpha 2b, an/or mixture thereof.

The above-described IBs were mixed with 1.5 L freshly prepared refolding buffer (100 mM Tris-HCl (pH 7.0), 0.5 M L-Arginine, 2 mM EDTA). The reaction mixture thus formed was incubated for 24˜36 hr. without stirring at room temperature to allow refolding of the recombinant Pro-hIFN alpha 2b, acetylated Pro-hIFN alpha 2b, an/or mixture thereof.

At this stage the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135 and/or acetylated tyrosine at position 136, in any other quantitative amount, ratio, or combinations thereof, indicates the present step of the manufacturing procedure was used to manufacture Pro-hIFN A2b. The detection of the presence of acetylated amino acids, and/or overall quantitative amount or any ratio before, during, or after any manufacturing step(s) of any part of the present disclosure's method was utilized to manufacture Pro-hIFN A2b. The protein can be denatured before refolding, and be folded after the folding conditions above.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b

### Example 4

### Ultrafiltration/Diafiltration

This method is not identical compared to known method of making Pro-hIFN A2b. Here the refolded mixture was centrifuged under about 10,000±1,000 rpm at room temperature. The collected supernatant was filtered through 0.2 μm filters and clarified solution was obtained. The filtrate was then concentrated 20 folds by a tangential Ultrafiltration system and diafiltered in constant-volume-mode with about 7 volumes of Q-A equilibrium buffer (about pH 7.0±0.1, conductivity<2 mS/cm). Finally, the retentate suspension was harvested. The suspension was then centrifuged at between about 8000-9,000 rpm, about 15-25 min at 4° C. The collected supernatant was then pumped through a Sartorius 0.2 μm filter. A volume of about 10-20 mL sample was taken out for protein concentration analysis by the BCA method. The filtrate was obtained and stored in refrigerator overnight at 4° C.

At this stage the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135 and/or acetylated tyrosine at position 136, in any other quantitative amount, ratio, or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b

### Example 5

### Ammonium Acetate/Sulfate Precipitation (AAP/ASP)

This example is another illustration of departure of known method of making Pro-hIFN alpha 2b. Here a buffer for ammonium acetate precipitation was prepared to contain about 48 g/L ammonium acetate (about 0.6 M NH₄OAc) and about 180 g/L sodium chloride at about pH 2.8. The precipitation was performed by adding 40 ml AAP buffer to Pro-hIFN alpha 2b, acetylated Pro-hIFN alpha 2b, an/or mixture thereof in glass bottle with stirring. The solution was then placed in 40° C. water bath and incubated for about 2 h. After incubation, the mixture was then clarified by centrifugation at about 9,000 rpm for about 20 min at 4° C. The pooled supernatant was adjusted to pH about 4.5. After the pooled supernatant was filtered by 0.2 μm filtration membrane, the filtrate was stored at 4° C. overnight.

At this stage the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135, and/or acetylated tyrosine at position 136, in any other quantitative amount, ratio, or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b

### Example 6

Purification (Anionic exchange, AIX)

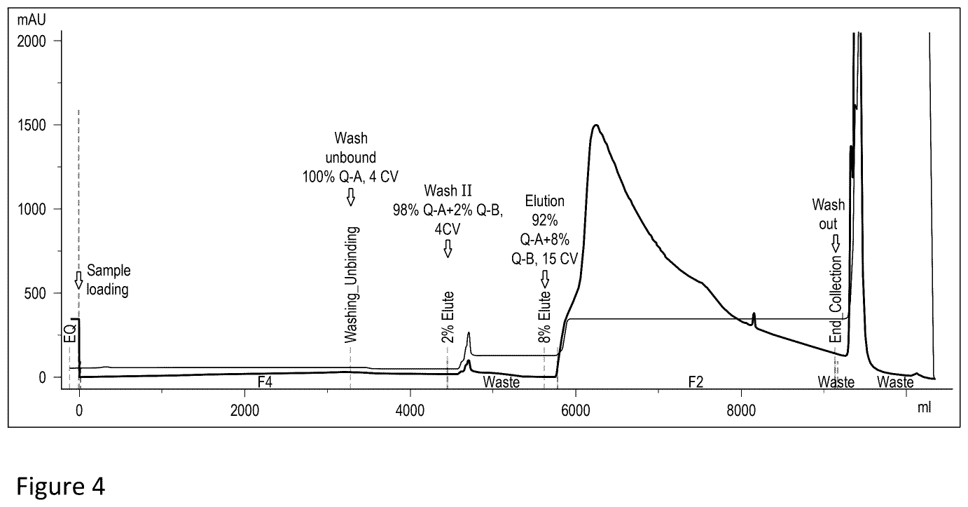

AIX column flow rate was at about 35 ml/min. there are two washing steps in before elution. The elution steps were performed at about 15 CV of elution buffer containing about 80 mM NaCl was used. The elution of Pro-hIFN alpha 2b, acetylated Pro-hIFN alpha 2b, an/or mixture thereof protein was all monitored by 280 nm UV detection. The wish condition is set at between 15-25 mM of wash salt solution, and the elution buffer has the salt concentration of between 75-85 mM. pHis maintained at about 7. The elution profile and steps of AIX purification performed was shown in FIG. 4.

At this stage the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135, and/or acetylated tyrosine at position 136, in any other quantitative amount, ratio, or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b

### Example 7

Peptide Mapping of Pro-IFN-α2b (and/or acetylated Pro-hIFN A2b)

Peptide mapping was employed to analyze the sequence of Pro-IFN alfa-2b (and/or acetylated Pro-hIFN A2b). Peptide maps were generated by the RP-HPLC separation of peptide mixtures resulting either from trypsin and endoproteinase Lys-C dual-digestion or from endoproteinase Glu-C digestion of a sample. After the digestion, the proteolytic reaction was quenched by the addition of trifluoroacetic acid (TFA). The resulting peptide mixtures were separated using RP-HPLC with TFA/acetonitrile (ACN) gradient to generate a non-reduced map. To obtain a reduced map, peptide mixtures were reduced with tris(2-carboxyethyl) phosphine hydrochloride (TCEP-HCl) after the reaction is quenched and then subjected to RP-HPLC. The eluted peptide peaks were monitored by UV detection and subsequently analyzed by on-line LC-MS/MS for detection of peptide masses and sequence of selected peptides.

### Trypsin/Lys-C Peptide Mapping

Table 1 summarizes the identification of all peptides obtained from the reduced trypsin/Lys-C peptide map. All expected peaks with respective sequence assignment and measurement of accurate masses are observed except for short peptide fragments that are not recoverable on the maps. These fragments contain only a single residue or dipeptides, such as T2, T4, T6, T12, T15, T16, T20, and T21; and some of them can be identified in partially digested long peptides such as T2-3, T6-7, T12-13 and T16-17.

TABLE 1

| | | | Monoisotopic | observed |
|---|---|---|---|---|
| Pepride | Residue | Sequence | Mass (cal.) | mass |
| T1 | 1-13 | PCDLPQTHSLGSE | 1409.67 | 1409.68 |
| T1a | 1-8 | PCDLPQTH | 909.40 | 989.41 |
| T2 | 14 | R | 174.11 | ND² |
| T3 | 15-23 | TLMLLAQMR | 1075.59 | 1075.60 |
| T4 | 24 | R | 174.11 | ND |
| | 25-32 | ISLFSCLK | 909.50 | 909.51 |
| T& | 33-34 | DR | 289.14 | ND |
| T7 | 35-50 | HDFGFPQEEFGNQFQK | 1953.86 | 1953.87 |
| T6-7 | 33-50 | DRHDFGFPQEEFGNQFQK | 2224.99 | 2225.00 |
| T8 | 51-73 | AETIPVLHEMIQQIFNLFSTK | 2458.29 | 2458.31 |
| T9 | 72-84 | DSSAAWDETLLDK | 1449.66 | 1449.67 |
| T10 | 85-113 | FYTELYQQLNDLEACVIQGVGVTETPLMK | 3301.63 | 3341.63 |
| T11 | 114-123 | EDSILAVR | 901.49 | 901.50 |
| T12 | 122 | K | 146.11 | ND |
| T13 | 123-126 | YFQR | 612.30 | 612.31 |
| T12-13 | 122-126 | KYFQR | 740.40 | 740.41 |
| T14 | 127-132 | ITLYLE | 749.47 | 749.48 |
| T15 | 133-134 | EK | 275.15 | ND |
| T16 | 135 | K | 146.11 | ND |
| T17 | 136-145 | YSPCAWEVVER | 1208.57 | 1208.58 |
| T16-17 | 134-145 | KYSPCAWEVVR | 1336.66 | 1336.67 |
| T18 | 146-150 | AFIMR | 618.32 | 618.32 |
| T19 | 151-163 | SFSLSTNLQESLR | 1480.75 | 1480.76 |
| T20 | 164-165 | SK | 133.14 | ND |
| T21 | 166 | E | 147.05 | ND |

T5

At this stage the mere fact that the detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135, and/or acetylated tyrosine at position 136 in any other quantitative amount, ratio, or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

### Example 8

### Unexpected Detection and Analysis of Acetylated Pro-IFN-α2b

Extracted ion chromatogram (EIC or XIC) was used and was created by plotting the intensity of the signal observed at chosen mass-to-charge value or series of values in a series of mass spectra recorded as a function of retention time. EIC allows the calculation of the content of the selected mixture component based on the value of the area under the peak. Total ion current chromatogram is obtained by plotting the total ion current detected in each of a series of mass spectra recorded as a function of retention time. Using extracted ion chromatogram (XIC) intensity ratio between modified and unmodified peptide known in the art, the following amino acid position as shown in Table 2 was determined to be acetylated.

TABLE 2
The quantification of the amount of acetylated Pro¹ and Lys⁵⁰ of Pro IFN A2B

| | | | | | overall |
|---|---|---|---|---|---|
| | Sequence | Theoretical | Experimental | Site of | quantitative |
| Peptide | Position | Mass (Da) | Mass (Da) | Modification | relative ratio |
| T1 | 1-13 | 1409.7 | 1409.7 | | 1 |
| Acetylated | | 1451.7 | 1451.7 | Pro 1 | |
| T1 | | | | | |
| T6-7-8 | 33-71 | 4665.3 | Not detected | | 1 |
| Acetylated | | 4707.3 | 4703.3 | Lys 50 | |
| T6-7-8 | | | | | |
| T11 | 114-121 | 901.5 | 901.5 | | 1 |
| Acetylated | | 943.5 | 943.5 | Ser 116 | |
| T11 | | | | | |
| T17 | 136-145 | 1208.6 | 1208.6 | | 5 |
| Acetylated | | 1250.6 | 1250.6 | Ser 137 | |
| T17 | | | | | |

shows their overall quantitative relative ratio is about 2:1. In the above table with quantification analysis, the acetylated Pro¹ and Lys⁵⁰ of Pro IFN A2B shows that their overall quantitative relative ratio amount is about 1:1.

The quantification amount of the acetylated Pro¹, Lys⁵⁰, and Ser¹¹⁶ Pro-hIFN alpha 2b's overall quantitative relative ratio is about 1:1:1. In another quantification analysis, the acetylated amount of Pro¹-acetylated Pro-hIFN alpha 2b to acetylated Lys⁵⁰ Pro-hIFN alpha 2b to acetylated Ser¹¹⁶ Pro-hIFN alpha 2b's overall quantitative relative ratio is about 2:1:1.

Table 2's quantification of the acetylated Pro¹, Lys⁵⁰, Ser¹¹⁶, and Ser¹³⁷ Pro-hIFN alpha 2b's shows their amount has an overall quantitative relative ratio of about 1:1:1:5. In another quantification analysis, the acetylated amount of Pro¹-acetylated Pro-hIFN alpha 2b to acetylated Lys⁵⁰ Pro-hIFN alpha 2b to acetylated Serin¹¹⁶ Pro-hIFN alpha 2b to acetylated Serine¹³⁷ Pro-hIFN alpha 2b has an overall quantitative relative ratio of about 2:1:1:5.

In one of the runs, acetylated lysine at position 32, acetylated lysine at position 113, acetylated lysine at position 132, and/or acetylated lysine at position 135 were detected. The ratio of acetylated Lys32:Lys113:Lys132:Lys135 is about 12:1:7.8:3.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

### Example 9

Ligand and Receptor Binding Assay for Pro-hIFN alpha 2b, acetylated Pro-hIFN alpha 2b, an/or mixture thereof.

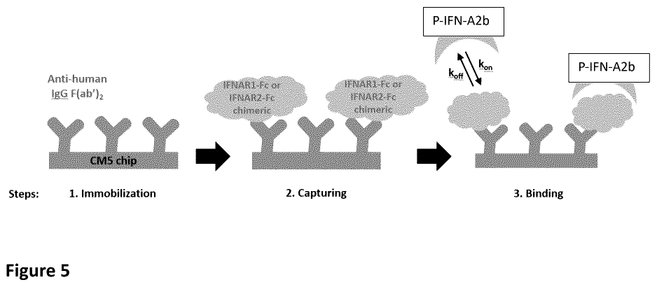

The canonical Type I IFN receptor consists of two chain receptors, IFNAR1 and IFNAR2. Interferon binds to the high binding affinity of IFNAR2 first and then IFNAR2 binds to the complex to initiate the downstream signaling pathways. Receptor binding activity of Pro-hIFN alpha 2b, acetylated Pro-hIFN alpha 2b, an/or mixture thereof were assessed by monitoring the binding of samples to human interferon alpha receptor using surface plasmon resonance (SPR) analysis. The analysis includes three steps: (1) immobilization of a capture antibody; (2) capture of IFNAR2-Fc-Fc; and (3) binding of Pro-hIFN alpha 2b, acetylated Pro-hIFN alpha 2b, an/or mixture thereof. The general concept is illustrated in FIG. 5.

In step 1, an anti-Human IgG (γ-chain specific) F(ab′) 2 fragment was covalently coupled to the carboxyl functionalized dextran layer on the transducer surface of CM5 sensor chip. In step 2, IFNAR1-Fc or IFNAR2-Fc fusion protein, consisting of the extracellular domain of human type I interferon receptor fused to Fc region of human IgG1, was then passed through the CM5 chip and captured by the immobilized F(ab′)2 fragment. In step 3, Pro-hIFN alpha 2b, acetylated Pro-hIFN alpha 2b, an/or mixture thereof samples at various concentrations were respectively injected over the chip surface containing captured IFNAR1-Fc or IFNAR2-Fc. As Pro-hIFN alpha 2b, acetylated Pro-hIFN alpha 2b, an/or mixture thereof binds to the captured receptor, an increase in SPR signal (expressed in response units, RU) is observed. After desired association time, a solution buffer without Pro-hIFN alpha 2b, acetylated Pro-hIFN alpha 2b, an/or mixture thereof is injected on the microfluidics that dissociates the bound complex. As Pro-hIFN alpha 2b, acetylated Pro-hIFN alpha 2b, an/or mixture thereof dissociates from the captured receptor, a decrease in SPR signal is observed. SPR analysis allows measurement of the binding kinetics (association and dissociation rate constants, kon and koff) and estimation of equilibrium dissociation constant (KD) for binding interaction between Pro-IFN alfa-2b and receptor.

Table 3 lists kon, koff, KD and R1xR2 values for both IFNAR1 and IFNAR2 obtained from Pro-IFN alfa-2b, acetylated Pro-IFN alfa-2b, and/or mixture thereof. R1xR2 represents the combined receptor binding affinity for the interferon chain receptors. Results show that they are comparable, and the determined KD is in the range of between about 3.27 to 7.45 μM for IFNAR1 and between about 1.15 to 1.79 nM for IFNAR2, indicating that Pro-IFN alfa-2b, acetylated Pro-IFN alfa-2b, and/or mixture thereof is able to effectively bind to human type I interferon receptor. Also, the difference of KD between IFNAR1 to IFNAR2 for Pro-IFN alfa-2b, acetylated Pro-IFN alfa-2b, and/or mixture thereof toward receptors is about ˜1000 fold, which is consistent with the published reference articles.

Table 3 shows Kinetic Parameters Determined for the Binding of Pro-IFN alfa-2b, acetylated Pro-IFN alfa-2b, and/or mixture thereof to IFNAR2-Fc.

TABLE 3

| | | | KD |
|---|---|---|---|
| Sample | kon(1/M · s) | koff (1/s) | (nM) |
| 1 | (1.339 ± 0.153)E+07 | (1.758 ± 0.035)E−02 | 1.33 |
| 2 | (1.183 ± 0.016)E+07 | (1.618 ± 0.013)E−02 | 1.37 |
| 3 | (1.098 ± 0.009)E+07 | (1.515 ± 0.009)E−02 | 1.38 |
| 4 | (9.598 ± 0.142)E+06 | (1.337 ± 0.015)E−02 | 1.39 |
| 5 | (1.266 ± 0.018)E+07 | (1.504 ± 0.018)E−02 | 1.19 |

Table 4 shows IFNAR binding kinetic and affinity measurements

TABLE 4

| | IFNAR1 | | | IFNAR2 | | |
|---|---|---|---|---|---|---|
| Sample | kon (1/M · s) | koff (1/s) | R1 (M) | kon (1/M · s) | koff (1/s) | R2 (M) |
| standard | (4.389 + 0.225)E+5 | 3.272 + 0.142 | (7.456 + 0.067)E−6 | (1.045 + 0.030)E+7 | (1.879 + 0.029)E−2 | (1.799 + 0.024)E−9 |
| 1 | (4.179 + 0.142)E+5 | 1.969 + 0.027 | (4.715 + 0.107)E−6 | (1.138 + 0.011)E+7 | (1.736 + 0.007)E−2 | (1.524 + 0.018)E−9 |
| 2 | (4.272 + 0.109)E+5 | 1.905 + 0.099 | (4.458 + 0.144)E−6 | (1.536 + 0.008)E+7 | (1.766 + 0.018)E−2 | (1.150 + 0.006)E−9 |
| 3 | (4.041 + 0.037)E+5 | 1.323 + 0.018 | (3.274 + 0.071)E−6 | (1.135 + 0.041)E+7 | (1.807 + 0.050)E−2 | (1.593 + 0.013)E−9 |
| 4 | (2.663 + 0.166)E+5 | 1.727 + 0.097 | (6.489 + 0.126)E−6 | (1.162 + 0.017)E+7 | (1.780 + 0.015)E−2 | (1.532 + 0.090)E−9 |
| 5 | (3.304 + 0.219)E+5 | 1.168 + 0.130 | (3.713 + 0.150)E−6 | (1.240 + 0.013)E+7 | (1.745 + 0.007)E−2 | (1.407 + 0.018)E−9 |

| | Combined R1 × R2 | Relative Combined |
|---|---|---|
| Sample | (μM × μM) | Affinity |
| standard | 0.0134 | 0.54 |
| 1 | 0.0072 | 1 |
| 2 | 0.0052 | 1.4 |
| 3 | 0.0052 | 1.38 |
| 4 | 0.0099 | 0.72 |
| 5 | 0.0052 | 1.38 |

Detection of existence of one or more acetylated amino acid selected from Proline at position 1 at the N-terminus, Lysine at position 50, Serine at position 116, and Serine at position 137 in any overall quantitative ratio, such as 1:1:1:5 or 2:1:1:5, and/or acetylated lysine at position 32, acetylated lysine at position 71, acetylated lysine at position 113, acetylated lysine at position 132, acetylated lysine at position 135 and/or acetylated tyrosine at position 136, in any other quantitative amount, ratio, or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

### Example 10

### Multiple Runs

Multiple runs of Pro-IFN alfa-2b were synthesized multiple times in accordance with the above examples.

In one of the multiple runs, the fermentation steps were carried out as disclosed in U.S. Patent Application Publication Number US20250101484A1, in particular, the fermentation was carrying out as: seeding (time S₀) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I₀ (time at initiation of induction) or I₁ or both; optionally, supplying a nitrogen source to any of the one or more fermenters, individually, at one or more time points selected from about S₅, S₆, S₇, S₈, S₉ and S₁₀. The mere existence of one or more of the acetylated species indicated in the present disclosure, shows that the present disclosure's method, in part or in whole was utilized.

In another one of the multiple runs, acetylation species were also detected with acetylated Lys at position 32, acetylated Lys at position 113, acetylated Lys at position 132, and acetylated Lys at position 135. The mere existence of one or more of the acetylated species indicated in the present disclosure, showed that the present disclosure's method, in part or in whole was utilized.

Some runs showed that the ratio between acetylated Lys at position 32, acetylated Lys at position 113, acetylated Lys at position 132, and acetylated Lys at position 135 is about 12:1:7.8:3. Alternatively, or additionally, the ratio was about 4:0.3:2.6:1. The mere existence of one or more of the acetylated species indicated in the present disclosure, showed that the present disclosure's method, in part or in whole was utilized.

In a run, a total of about 4-5% of acetylated species were produced as compare to non-acetylated Pro-hIFN A2b.

In another run, about 7.33 g of NAM was added during fermentation. Optionally, the temperature was elevated to about 40° C.

In another one of the multiple runs, acetylation species were also detected with acetylated Lys at position 71 and acetylation of tyrosine at position 136. The mere existence of one or more of the acetylated species indicated in the present disclosure, showed that the present disclosure's method, in part or in whole was utilized.

Some runs showed that the ratio between acetylated Lys at position 71 and acetylation of tyrosine at position 136 was about 1:28. The mere existence of one or more of the acetylated species indicated in the present disclosure, showed that the present disclosure's method, in part or in whole was utilized.

In another one of the multiple runs, acetylation species were also detected with acetylated Lys at position 50, acetylated Lys at position 71 and acetylation of tyrosine at position 136. The mere existence of one or more of the acetylated species indicated in the present disclosure, showed that the present disclosure's method, in part or in whole was utilized.

Additionally, at this stage the mere fact that the detection of existence of one or more acetylated amino acid lysine at position 32, acetylated lysine at position 71, acetylated lysine at position 113, acetylated lysine at position 132, acetylated lysine at position 135 and/or acetylated tyrosine at position 136 in other amount or combinations thereof, indicates the present step of the manufacturing procedure was used to manufacture Pro-hIFN A2b. The detection of the presence of acetylated amino acids, and/or overall quantitative any ratio before, during, or after any manufacturing step(s) of the present disclosure was utilized to manufacture Pro-hIFN A2b.

Some runs showed that the ratio between with acetylated Lys at position 50, acetylated Lys at position 71 and acetylation of tyrosine at position 136 was about 1.14:3.25:1. The mere existence of one or more of the acetylated species indicated in the present disclosure, showed that the present disclosure's method, in part or in whole was utilized.

In another one of the multiple runs, acetylation species were also detected with acetylated Lys at position 50, acetylated Lys at position 71, acetylated Lys at position 113, and acetylation of tyrosine at position 136. The mere existence of one or more of the acetylated species indicated in the present disclosure, showed that the present disclosure's method, in part or in whole was utilized.

Some runs showed that the ratio between with acetylated Lys at position 50, acetylated Lys at position 71, acetylated Lys at position 113, and acetylation of tyrosine at position 136 was about 32:1:91:28. The mere existence of one or more of the acetylated species indicated in the present disclosure, showed that the present disclosure's method, in part or in whole was utilized.

In another one of the multiple runs, acetylation species of Lys at position 50 is as high as 3.2%.

In another one of the multiple runs, acetylation species of Lys at position 113 is as high as 9.1%.

In another one of the multiple runs, acetylation species of Tyrosine at position 136 is as high as 2.8%.

In one run, the total acetylated species of Pro hIFN alpha 2b was as high as 50%. The weight was about 1.2 mg.

In one run, no acetylated species are detected.

It is contemplated that any embodiment discussed in this specification can be implemented with respect to any method, kit, reagent, or composition of the disclosure, and vice versa. Furthermore, compositions of the disclosure can be used to achieve methods of the disclosure.

It will be understood that particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What we claim are:

1. A composition comprising Pro-hIFN alpha 2b, acetylated Pro-hIFN alpha 2b, and/or mixture thereof.

2. The composition according to claim 1, wherein said acetylated Pro-hIFN alpha 2b comprises one or more acetylated positions from acetylated proline at position 1, acetylated serine at position 116, acetylated serine at position 137, and/or acetylated lysine at position 50 of SEQ ID NO: 2.

3. The composition according to claim 1, wherein said acetylated Pro-hIFN alpha 2b comprises one or more acetylated positions of acetylated lysine at position 32, acetylated lysine at position 113, acetylated lysine at position 132, and/or acetylated lysine at position 135 of SEQ ID NO: 2.

4. The composition according to claim 1, wherein said acetylated Pro-hIFN alpha 2b comprises one or more acetylated positions of acetylated lysine at position 71, and/or acetylated tyrosine at position 136 of SEQ ID NO: 2.

5. The composition according to claim 1, wherein said acetylated Pro-hIFN alpha 2b comprises one or more acetylated positions from acetylated proline at position 1, acetylated serine at position 116, acetylated serine at position 137, acetylated lysine at position 50, acetylated lysine at position 32, acetylated lysine at position 113, acetylated lysine at position 132, acetylated lysine at position 135, acetylated lysine at position 71, and/or acetylated tyrosine at position 136 of SEQ ID NO: 2.

6. The composition according to claim 1, wherein the composition comprises E. coli lysate containing inclusion bodies, and wherein the composition is obtained by a process including fermenting a culture of E. coli engineered to express Pro-hIFN alpha 2b at a condition including at between about 36.9° C. to 37.1° C. with dissolved oxygen at about 40%, at between about 40% to 45%, and/or above 45%;
wherein optionally, the E. coli comprises BLR-(DE 3)-RIL.

7. The composition according to claim 1, wherein the composition comprises an E. coli culture supernatant.

8. The composition according to claim 1, wherein the Pro-hIFN alpha 2b and/or acetylated Pro-hIFN alpha 2b is denatured.

9. The composition according to claim 1, wherein the composition comprises folded Pro-hIFN alpha 2b and/or folded acetylated Pro-hIFN alpha 2b.

10. A composition according to claim 1, wherein the composition is obtained by a process including mixing an E. coli culture supernatant containing denatured Pro-hIFN alpha 2b and/or denatured acetylated Pro-hIFN alpha 2b and a refolding buffer, and wherein the E. coli culture supernatant is produced by a procedure including fermenting a culture of E. coli engineered to express Pro-hIFN alpha 2b at a condition including at between 36.9 and 37.5° C. with dissolved oxygen at about 40%, between about 40% to 45%, and/or above 45%;
wherein optionally, the E. coli is BLR-(DE 3)-RIL.

11. The composition according to claim 10, wherein the process of obtaining the composition further includes one or more filtration steps, one or more ammonium acetate precipitation steps, and/or anion exchange chromatography step.

12. The composition according to claim 10, wherein the pH of the composition or during the process of obtaining the composition is maintained at between about 6.9 to 7.4 or below.

13. The composition according to claim 1, wherein said acetylated Pro-hIFN alpha 2b comprises two, three, four, or more acetylated positions.

14. A pharmaceutical formulation comprising the composition according to claim 1; and
one ore more pharmaceutically acceptable carrier.

15. The composition according to claim 2,
(a) wherein overall quantitative relative ratio amount of the Pro1-acetylated Pro-hIFN alpha 2b to acetylated Lys50 Pro-hIFN alpha 2b is about 2:1;
wherein overall relative ratio amount of the Pro1-acetylated Pro-hIFN alpha 2b to acetylated Lys50 Pro-hIFN alpha 2b is about 1:1;
wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 1:1:1;
wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 2:1:1;
wherein overall quantitative relative ratio of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b is about 1:1:1:5; or
wherein overall quantitative relative ratio amount of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b is about 2:1:1:5.

16. The composition according to claim 3,
wherein overall quantitative relative ratio amount of acetylated Lys32:Lys113 is about 7.8:1; 10:1, 11:1 or 12:1;
wherein overall quantitative relative ratio amount of acetylated Lys132:Lys135 is about 2.6:1, 7:3, or 8:3; or
wherein overall quantitative relative ratio amount of the ratio of acetylated Lys32:Lys113:Lys132:Lys135 is about 12:1:7.8:3, and/or 4:0.3:2.6:1.

17. The composition according to claim 5,
wherein overall quantitative relative ratio amount of acetylated Lys71:Tyr136 is about 1:28, or 1:30;
wherein overall quantitative relative ratio amount of acetylated Lys50:Lys71:Lys113:Try 136 is about 32:1:91:28;
wherein overall quantitative relative ratio amount of the ratio of acetylated Lys50:Lys113:Try 136 is about 1.14:3.25:1.

18. A method for effectively treating a subject in need who has a disorder including myelofibrosis, polycythemia vera, essential thrombocythaemia, and/or chronic myeloid leukemia comprising administrating to the subject a therapeutically effective amount of the composition according to claim 1.

19. The method according to claim 18, wherein the myelofibrosis comprises idiopathic, primary, and/or early myelofibrosis.

20. The method according to claim 18, wherein the polycythemia vera comprises high and/or low risk polycythemia vera.

21. A method for producing the composition according to claim 1, the method comprising:
(a) cloning a vector using DNA encoding the amino acid sequence of Pro-hIFN alpha 2b;
(b) transfecting said vector into a host cell
(c) fermenting said host cell at a condition including at about 37° C. with a dissolved oxygen at about 40%, between about 40% to 45%, and/or above 45%;
(d) inducing Pro-hIFN alpha 2b expression with IPTG at a concentration of between about 0.5-0.9 mM;
(e) removing the initial methionine from said Pro-hIFN alpha 2b;
(f) refolding said Pro-hIFN alpha 2b;
(g) performing ultrafiltration on said Pro-hIFN protein;
(h) performing amino acetate precipitation on said Pro-hIFN alpha 2b; and
(i) purifying said Pro-hIFN alpha 2b.

22. The method for producing the composition according to claim 21, wherein said fermenting in said step (c) comprises:
carrying out seeding (time S₀) of a culture of prokaryote host cell engineered to inducibly express a recombinant protein and transferring the culture into one or more fermenters each containing a culture medium;
measuring the dissolved oxygen (DO) level, an agitation rate, and a pH in the one or more fermenters;
supplying a carbon source feeding solution to any of the one or more fermenters individually, whenever condition(s) (i) and/or (ii) are met:
(i) said DO level exceeds above about 35% to 45% and said agitation rate exceeds about 300 to 1,000 rpm;
(ii) said agitation rate exceeds about 400 to 700 rpm and said pH exceeds about 7.0 to 7.4; and
supplying a nitrogen source to any of the one or more fermenters in which induction of expression of the recombinant protein has been initiated, individually, at about I₀ (time at initiation of induction) or I₁ or both;
optionally, supplying a nitrogen source to any of the one or more fermenters, individually, at one or more time points selected from about S₅, S₆, S₇, S₈, S₉ and S₁₀.

23. The method according to claim 21 further comprises the step of detecting and/or quantifying one or more acetylated Pro-hIFN alpha 2b.

24. The method according to claim 22, further comprises the step of quantifying detecting and/or quantifying one or more acetylated Pro-hIFN alpha 2b.

25. A method of detecting whether a manufacturing procedure is used to produce Pro-hIFN alpha 2b, said method comprises
analyzing and/or quantifying the present of one or more acetylated Pro-hIFN alpha 2b in a product, a product batch, or an intermediate thereof from said product, product batch or intermediate from a manufacturing procedure.

26. The method according to claim 25, wherein said acetylated Pro-hIFN alpha 2b comprises one or more acetylated proline at position 1, acetylated serine at position 116, acetylated serine at position 137, and/or acetylated lysine at position 50 of said Pro-hIFN alpha.

27. The method according to claim 25, wherein said acetylated Pro-hIFN alpha 2b comprises one or more acetylated positions from acetylated lysine at position 32, acetylated lysine at position 113, acetylated lysine at position 132, and/or acetylated lysine at position 135 of said Pro-hIFN alpha.

28. The method according to claim 25, wherein said acetylated Pro-hIFN alpha 2b comprises acetylated lysine at position 71 and/or acetylated tyrosine at position 136 of said Pro-hIFN alpha.

29. The method according to claim 23, wherein the presence of said one or more acetylated Pro-hIFN alpha 2b is analyzed in one or more samples obtained before, during, or after one or more steps in a manufacturing procedure of claim 21, wherein the presence of said one or more acetylated Pro-hIFN alpha 2b indicates one or more of the steps in the manufacturing procedure of claim 21 was used.

30. The method according to claim 24, wherein the presence of said one or more acetylated Pro-hIFN alpha 2b is analyzed in one or more samples obtained before, during, or after one or more steps in a manufacturing procedure of claim 22, wherein the presence of said one or more acetylated Pro-hIFN alpha 2b indicates one or more of the steps in the manufacturing procedure of claim 22 was used.

31. The method according to claim 23, wherein the step of detecting and/or quantifying the presence of acetylated Pro-hIFN alpha 2b is performed in products and/or intermediates from one or more individual batches of a manufacturing procedure according to claim 21.

32. The method according to claim 24, wherein the step of detecting and/or quantifying the presence of acetylated Pro-hIFN alpha 2b is performed in products and/or intermediates from one or more individual batches of a manufacturing procedure according to claim 22.

33. The method according to claim 23, wherein the presence of any one or more of the following overall quantitative relative ratio amount of acetylated Pro-hIFN alpha 2b indicates any of the one or more steps of the method of claim 21 was used:
(a) wherein overall quantitative relative ratio amount of the Pro1-acetylated Pro-hIFN alpha 2b to acetylated Lys50 Pro-hIFN alpha 2b is about 2:1;
(b) wherein overall relative ratio amount of the Pro1-acetylated Pro-hIFN alpha 2b to acetylated Lys50 Pro-hIFN alpha 2b is about 1:1;
(c) wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 1:1:1;
(d) wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 2:1:1;
(e) wherein overall quantitative relative ratio of Pro1-acetylated Pro-hIFN alpha 2b to acetylated Lys50 Pro-hIFN alpha 2b to acetylated Serin116 Pro-hIFN alpha 2b to acetylated Serine 137 Pro-hIFN alpha 2b is about 1:1:1:5;
(f) wherein overall quantitative relative ratio amount of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b is about 2:1:1:5;
(g) wherein overall quantitative relative ratio amount of acetylated Lys32:Lys113:Lys132:Lys135 Pro-hIFN alpha 2b is about 12:1:7.8:3;
(h) wherein overall quantitative relative ratio amount of acetylated Lys32:Lys113 of Pro-hIFN alpha 2b is between about 10:1, 11:1 or 12:1;
(i) wherein overall quantitative relative ratio amount of acetylated Lys132:Lys135 of Pro-hIFN alpha 2b is about 2:1, 7:3, or 8:3;
(j) wherein overall quantitative relative ratio amount of acetylated Lys32:Lys113 of Pro-hIFN alpha 2b is about 7.8:1; 10:1, 11:1 or 12:1;
(k) wherein overall quantitative relative ratio amount of acetylated Lys132:Lys135 of Pro-hIFN is about 2.6:1, 7:3, or 8:3;
(l) wherein overall quantitative relative ratio amount of the ratio of acetylated Lys32:Lys113:Lys132:Lys135 of Pro-hIFN is about 12:1:7.8:3, and/or 4:0.3:2.6:1;
(m) wherein overall quantitative relative ratio amount of acetylated Lys71:Tyr136 of Pro-hIFN is about 1:28, or 1:30;
(n) wherein overall quantitative relative ratio amount of acetylated Lys50:Lys71:Lys 113:Try 136 of Pro-hIFN is about 32:1:91:28; and/or
(o) wherein overall quantitative relative ratio amount of the ratio of acetylated Lys50:Lys113:Try 136 is about 1.14:3.25:1.

34. The method according to claim 24, wherein the presence of any one or more of the following overall quantitative relative ratio amount of acetylated Pro-hIFN alpha 2b indicates any of the one or more steps of the method of claim 22 was used:
(a) wherein overall quantitative relative ratio amount of the Pro1-acetylated Pro-hIFN alpha 2b to acetylated Lys50 Pro-hIFN alpha 2b is about 2:1;
(b) wherein overall relative ratio amount of the Pro1-acetylated Pro-hIFN alpha 2b to acetylated Lys50 Pro-hIFN alpha 2b is about 1:1;
(c) wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 1:1:1;
(d) wherein overall quantitative relative ratio amount of the Pro1-acetylated Pro-hIFN alpha 2b to acetylated Lys50 Pro-hIFN alpha 2b to acetylated Serin116 Pro-hIFN alpha 2b is about 2:1:1;
(e) wherein overall quantitative relative ratio of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b is about 1:1:1:5;
(f) wherein overall quantitative relative ratio amount of Pro1-acetylated Pro-hIFN alpha 2b to acetylated Lys50 Pro-hIFN alpha 2b to acetylated Serin116 Pro-hIFN alpha 2b to acetylated Serine137 Pro-hIFN alpha 2b is about 2:1:1:5;
(g) wherein overall quantitative relative ratio amount of acetylated Lys32:Lys113:Lys132:Lys135 Pro-hIFN alpha 2b is about 12:1:7.8:3;
(h) wherein overall quantitative relative ratio amount of acetylated Lys32:Lys113 of Pro-hIFN alpha 2b is between about 10:1, 11:1 or 12:1;
(i) wherein overall quantitative relative ratio amount of acetylated Lys132:Lys135 of Pro-hIFN alpha 2b is about 2:1, 7:3, or 8:3;
(j) wherein overall quantitative relative ratio amount of acetylated Lys32:Lys113 of Pro-hIFN alpha 2b is about 7.8:1; 10:1, 11:1 or 12:1;
(k) wherein overall quantitative relative ratio amount of acetylated Lys132:Lys135 of Pro-hIFN is about 2.6:1, 7:3, or 8:3;
(l) wherein overall quantitative relative ratio amount of the ratio of acetylated Lys32:Lys113:Lys132:Lys135 of Pro-hIFN is about 12:1:7.8:3, and/or 4:0.3:2.6:1;
(m) wherein overall quantitative relative ratio amount of acetylated Lys71:Tyr136 of Pro-hIFN is about 1:28, or 1:30;
(n) wherein overall quantitative relative ratio amount of acetylated Lys50:Lys71:Lys113:Try 136 of Pro-hIFN is about 32:1:91:28; and/or
(o) wherein overall quantitative relative ratio amount of the ratio of acetylated Lys50:Lys 113:Try 136 is about 1.14:3.25:1.

35. The method according to claim 23, wherein the presence of any one or more of the following overall quantitative relative ratio amount of acetylated Pro-hIFN alpha 2b indicates the method of claim 21 was used:
(a) wherein overall quantitative relative ratio amount of acetylated Lys32:Lys113:Lys132:Lys135 Pro-hIFN alpha 2b is about 12:1:7.8:3;
(b) wherein overall quantitative relative ratio amount of acetylated Lys32:Lys113 of Pro-hIFN alpha 2b is between about 10:1, 11:1 or 12:1;
(c) wherein overall quantitative relative ratio amount of acetylated Lys132:Lys135 of Pro-hIFN alpha 2b is about 2:1, 7:3, or 8:3;
(d) wherein overall quantitative relative ratio amount of acetylated Lys32:Lys113 of Pro-hIFN alpha 2b is about 7.8:1; 10:1, 11:1 or 12:1;
(e) wherein overall quantitative relative ratio amount of acetylated Lys132:Lys135 of Pro-hIFN is about 2.6:1, 7:3, or 8:3;
(f) wherein overall quantitative relative ratio amount of the ratio of acetylated Lys32:Lys113:Lys132:Lys135 of Pro-hIFN is about 12:1:7.8:3, and/or 4:0.3:2.6:1; and/or
(g) wherein overall quantitative relative ratio amount of acetylated Lys71:Tyr136 of Pro-hIFN is about 1:28, or 1:30.

36. The method according to claim 24, wherein the presence of any one or more of the following overall quantitative relative ratio amount of acetylated Pro-hIFN alpha 2b indicates the method of claim 22 was used:
(a) wherein overall quantitative relative ratio amount of acetylated Lys32:Lys113:Lys132:Lys135 Pro-hIFN alpha 2b is about 12:1:7.8:3;
(b) wherein overall quantitative relative ratio amount of acetylated Lys32:Lys113 of Pro-hIFN alpha 2b is between about 10:1, 11:1 or 12:1;
(c) wherein overall quantitative relative ratio amount of acetylated Lys132:Lys135 of Pro-hIFN alpha 2b is about 2:1, 7:3, or 8:3;
(d) wherein overall quantitative relative ratio amount of acetylated Lys32:Lys113 of Pro-hIFN alpha 2b is about 7.8:1; 10:1, 11:1 or 12:1;
(e) wherein overall quantitative relative ratio amount of acetylated Lys132:Lys135 of Pro-hIFN is about 2.6:1, 7:3, or 8:3;
(f) wherein overall quantitative relative ratio amount of the ratio of acetylated Lys32:Lys113:Lys132:Lys135 of Pro-hIFN is about 12:1:7.8:3, and/or 4:0.3:2.6:1; and/or
(g) wherein overall quantitative relative ratio amount of acetylated Lys71:Tyr136 of Pro-hIFN is about 1:28, or 1:30.


SEQUENCE LISTING (2 sequences)

SEQ ID NO: 1 - protein, 165 aa, Synthetic construct [sequence omitted]

SEQ ID NO: 2 - protein, 166 aa, Synthetic construct
PCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE